(19) United States
(12) Patent Application Publication
(10) Pub. No.: US 2025/0149179 A1
(43) Pub. Date: May 8, 2025

(54) WORK ASSISTING APPARATUS, WORK ASSISTING METHOD, AND NON-TRANSITORY COMPUTER-READABLE STORAGE MEDIUM

(71) Applicant: NEC Corporation, Minato-ku, Tokyo (JP)

(72) Inventor: Hirotaka MAESHIMA, Tokyo (JP)

(73) Assignee: NEC Corporation, Minato-ku, Tokyo (JP)

(21) Appl. No.: 18/835,503

(22) PCT Filed: Mar. 18, 2022
(86) PCT No.: PCT/JP2022/012914

§ 371 Date: Aug. 2, 2024

(51) Int. Cl. G16H 50/30; G06V 10/70; G06V 40/16

(52) U.S. Cl. CPC G16H 50/30; G06V 10/70; G06V 40/174; G06V 40/178

(57) ABSTRACT

A work assisting apparatus (100) includes an acquisition unit (102) that acquires biological information of a user and resume information of the user, a computation unit (104) that computes an evaluation value indicating a health risk for the user, by using the biological information and the resume information that have been acquired, and a determination unit (106) that determines, by using the evaluation value indicating the health risk, a candidate of a task to present to the user.

### TECHNICAL FIELD

The present invention relates to a work assisting apparatus, a work assisting method, and a storage medium.

### BACKGROUND ART

Patent Document 1 describes one example of a health management system with work assistance capable of meeting needs of a user who desires work assistance, such as an elderly person. The system of Patent Document 1 is a health management system in which a user terminal and a main server are connected through a network line, wherein the main server includes a database that registers user information, a work ability score evaluation unit that evaluates a work ability score of an individual user, a health state score evaluation unit that evaluates a health state score of an individual user, a program provision unit that provides a work assisting program and a health improvement program, a work course certification unit that certifies a work course of an individual user, a work certification information provision unit that provides work certification information of the user to a work destination, and a point return unit that returns a work point based on work performance. This configuration enables the system described in Patent Document 1 to lead work assistance for an elderly person to learning assistance for a child.

### RELATED DOCUMENT

### Patent Document

Patent Document 1: Japanese Patent Application Publication No. 2019-175447

### SUMMARY OF INVENTION

### Technical Problem

In a system described in Patent Document 1 described above, a state of a user is evaluated by using a work ability score and a health state score. The work ability score is generated by a system administrator by inputting evaluation of a user for an evaluation item from an input screen of a work assisting site, and a health state score is also generated by the user by inputting interview evaluation or an actual measurement result from an input screen of an online health interview assisting site. Thus, a technique described in Patent Document 1 has a problem that evaluation requires manual data input in advance and is time-consuming, and processing of the system is also complicated and high in load.

In view of the problem described above, one example of an object of the present invention is to provide a work assisting apparatus, a work assisting method, and a storage medium that can assist getting employment suited to a health state in a simple process.

### Solution to Problem

According to one aspect of the present invention, there is provided a work assisting apparatus including:
an acquisition unit that acquires biological information of a user and resume information of the user;
a computation unit that computes an evaluation value indicating a health risk for the user, by using the biological information and the resume information that have been acquired; and
a determination unit that determines, by using the evaluation value indicating the health risk, a candidate of a task to present to the user.

According to one aspect of the present invention, there is provided a work assisting method including,
by one or more computers:
acquiring biological information of a user and resume information of the user;
computing an evaluation value indicating a health risk for the user, by using the biological information and the resume information that have been acquired; and
determining, by using the evaluation value indicating the health risk, a candidate of a task to present to the user.

According to one aspect of the present invention, there is provided a computer-readable storage medium storing a program that causes a computer to execute:
a procedure of acquiring biological information of a user and resume information of the user;
a procedure of computing an evaluation value indicating a health risk for the user, by using the biological information and the resume information that have been acquired; and
a procedure of determining, by using the evaluation value indicating the health risk, a candidate of a task to present to the user.

Note that, another aspect of the present invention may be a program that causes at least one or more computers to execute the method according to the one aspect described above, or may be a computer-readable storage medium storing such a program. The storage medium includes a non-transitory tangible medium.

The computer program includes a computer program code that, when executed by a computer, causes the computer to execute the work assisting method on a work assisting apparatus.

Note that, any combination of the above components, and conversion of an expression according to the present invention among a method, an apparatus, a system, a storage medium, a computer program, and the like is also effective as an aspect of the present invention.

Moreover, various components according to the present invention do not necessarily need to exist individually and independently, and may be formation of a plurality of components as one member, formation of one component with a plurality of members, a certain component being a part of another component, overlap of a part of a certain component and a part of another component, or the like.

Moreover, although a plurality of procedures are described in order in a method and a computer program according to the present invention, an order of the description does not limit an order of executing a plurality of procedures. Thus, when the method and the computer program according to the present invention are implemented, an order of the plurality of procedures can be changed to an extent that content does not contradict.

Further, a plurality of procedures of the method and the computer program according to the present invention are not limited to being executed at individually different timings. Thus, it may be occurrence of another procedure during execution of a certain procedure, partial or entire overlap of execution timing of a certain procedure and execution timing of another procedure, or the like.

### Advantageous Effects of Invention

According to one aspect of the present invention, a work assisting apparatus, a work assisting method, and a storage medium that assist getting employment suited to a health state in a simple process.

### BRIEF DESCRIPTION OF THE DRAWINGS

FIG. 1 It is a diagram illustrating an outline of a work assisting apparatus according to an example embodiment.

FIG. 2 It is a flowchart illustrating an operation example of the work assisting apparatus according to the present example embodiment.

FIG. 3 It is a diagram conceptually illustrating a system configuration of a work assisting system according to an example embodiment.

FIG. 4 It is a block diagram illustrating a hardware configuration of a computer that achieves a work assisting apparatus according to the example embodiment.

FIG. 5 It is a functional block diagram illustrating a functional configuration example of a work assisting apparatus according to the example embodiment.

FIG. 6 It is a diagram illustrating a data structure example of biological information and resume information of a user.

FIG. 7 It is a diagram illustrating a data structure example of health risk information.

FIG. 8 It is a diagram illustrating a data structure example of a task item list.

FIG. 9 It is a flowchart illustrating an operation example of the work assisting apparatus according to the example embodiment.

FIG. 10 It is a diagram illustrating one example of a task candidate screen.

FIG. 11 It is a diagram illustrating a data structure example of health risk information.

FIG. 12 It is a diagram illustrating a data structure example of a task item list.

FIG. 13 It is a flowchart illustrating an operation example of a work assisting apparatus according to an example embodiment.

FIG. 14 It is a diagram illustrating a data structure example of health risk information according to the present example embodiment.

FIG. 15 It is a flowchart illustrating an operation example of a work assisting apparatus according to the example embodiment.

FIG. 16 It is a functional block diagram illustrating a functional configuration example of a work assisting apparatus according to an example embodiment.

FIG. 17 It is a diagram illustrating a data structure example of a remuneration item list.

FIG. 18 It is a flowchart illustrating an operation example of a work assisting apparatus according to the example embodiment.

FIG. 19 It is a flowchart illustrating an operation example of a work assisting apparatus according to an example embodiment.

FIG. 20 It is a diagram illustrating a data structure example of help information.

FIG. 21 It is a functional block diagram illustrating another functional configuration example of a work assisting apparatus according to the example embodiment.

### EXAMPLE EMBODIMENT

Hereinafter, example embodiments of the present invention are described by using the drawings. Note that, a similar reference sign is assigned to a similar component in all the drawings, and description is not included as appropriate. Moreover, in each of the following figures, a configuration of a part that does not concern essence of the present invention is not included and not illustrated.

In the example embodiment, “acquisition” includes at least one of fetching, by a local apparatus, data or information stored in another apparatus or a storage medium (active acquisition), and inputting, into a local apparatus, data or information output from another apparatus (passive acquisition). Examples of active acquisition include requesting or inquiring of the another apparatus and receiving a reply thereof, accessing the another apparatus or the storage medium and reading, and the like. Moreover, an example of passive acquisition includes receiving information given by distribution (or transmission, push notification, or the like), and the like. Further, “acquisition” may include selecting and acquiring from received data or information, or selecting and receiving distributed data or information.

### <Minimum Configuration Example>

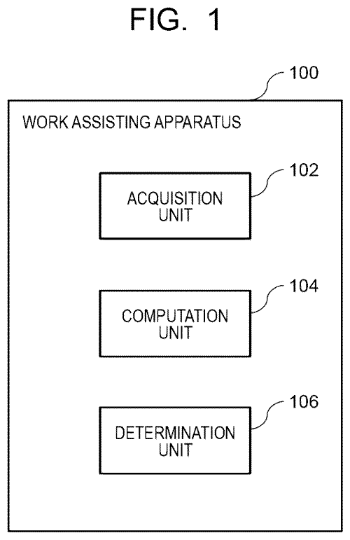

FIG. 1 is a diagram illustrating an outline of a work assisting apparatus 100 according to an example embodiment. The work assisting apparatus 100 includes an acquisition unit 102, a computation unit 104, and a determination unit 106.

The acquisition unit 102 acquires biological information of a user 20 and resume information of the user.

The computation unit 104 computes an evaluation value indicating a health risk for the user, by using the biological information and the resume information that have been acquired. The determination unit 106 determines, by using the evaluation value indicating the health risk, a candidate of a task to present to the user.

### <Operation Example>

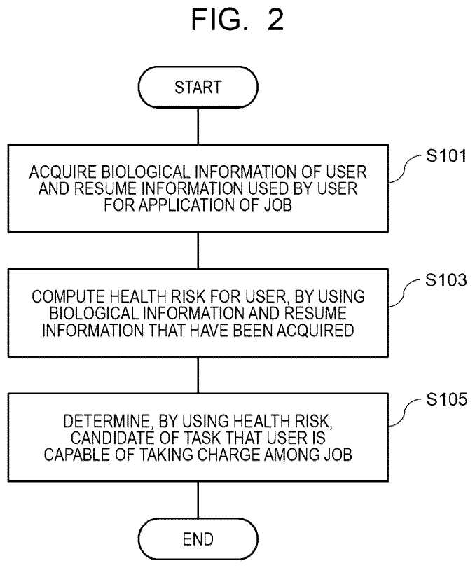

FIG. 2 is a flowchart illustrating an operation example of the work assisting apparatus 100 according to the present example embodiment.

First, the acquisition unit 102 acquires biological information of a user and resume information of the user (step S101).

Then, the computation unit 104 computes an evaluation value indicating a health risk for the user, by using the biological information and the resume information that have been acquired (step S103).

Then, the determination unit 106 determines, by using the evaluation value indicating the health risk, a candidate of a task to present to the user (step S105).

According to the work assisting apparatus 100, the acquisition unit 102 acquires biological information of a user and resume information of the user, the computation unit 104 computes an evaluation value indicating a health risk for the user, by using the biological information and the resume information that have been acquired, and the determination unit 106 determines, by using the evaluation value indicating the health risk, a candidate of a task to present to the user. Thereby, the user is assigned a job. Thus, when the work assisting apparatus 100 is used, work suited to a health state can be appropriately assisted in a simple process.

A detailed example of the work assisting apparatus 100 is described below.

### First Example Embodiment

### <System Outline>

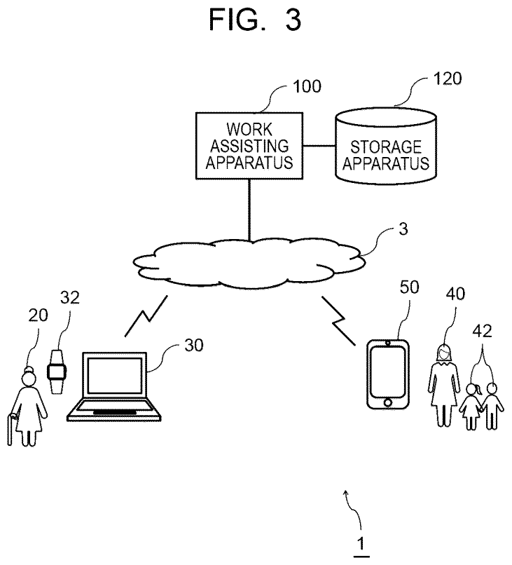

FIG. 3 is a diagram conceptually illustrating a system configuration of a work assisting system 1 according to an example embodiment.

The work assisting system 1 includes a work assisting apparatus 100. The work assisting apparatus 100 includes a storage apparatus 120. The storage apparatus 120 may be provided inside the work assisting apparatus 100, or may be provided outside the work assisting apparatus 100. In other words, the storage apparatus 120 may be hardware integrated with the work assisting apparatus 100, or may be hardware separate from the work assisting apparatus 100.

The work assisting apparatus 100 is, for example, a server computer, and may further include a web server.

The work assisting apparatus 100 can include a user terminal 30 and a wearable terminal 32 that are connected via a communication network 3. Further, the work assisting apparatus 100 may include an assistance target person terminal 50 connected via the communication network 3. The user terminal 30 is a terminal operated by the user 20, and the assistance target person terminal 50 is a terminal operated by an assistance target person 40.

The wearable terminal 32 is worn by the user 20, acquires biological information of the user 20, and transmits the acquired biological information to the work assisting apparatus 100 via the user terminal 30 or directly. When via the user terminal 30, for example, the wearable terminal 32 transmits the biological information to the user terminal 30 by near field communication (NFC). Moreover, the wearable terminal 32 is wristwatch-shaped in the figure, but is not limited thereto. Moreover, by using the user terminal 30 equipped with a camera, being a smartphone or the like, of the user 20, an image captured with the camera of the user terminal 30 may be analyzed by image processing by using a predetermined application or the like, and biological information may be estimated and transmitted to the work assisting apparatus 100. The biological information estimated herein includes, for example, a heart rate, a body temperature, a blood pressure, a respiratory rate, a stress level, and the like, but is not limited to thereto.

The user 20 performs a job of assisting the assistance target person 40. The user 20 is a person who has experience in at least one of housework and child rearing. The user 20 is a person in a non-working state. The user 20 is, for example, preferably 50 years old or older, and at least at equal to or more than an age at which osteoporosis development and dementia development begin to be seen (elderly person). However, an age of the user 20 may be younger than 50 years. Moreover, the user 20 can be said to be an elderly person who has not yet become ill, but is elderly, and can therefore be considered to have a potential health risk. Thus, when assisting getting work, it is preferable to assign a task with a task load suited to a health risk.

Moreover, the user 20 is not limited to an elderly woman, and may include a third assister (a person dispatched from a government, a company, an organization, or the like) who sympathizes with or approves of such an effort.

The assistance target person 40 is a person who desires assistance of child rearing. The assistance target person 40 is, particularly, a parent who has at least one child 42. Further, the assistance target person 40 is a parent of a single-parent family. Further, the assistance target person 40 is a woman, and is a mother of a single-parent household, a so-called single mother. Moreover, the assistance target person 40 may include a parent of a family who is living separately and whose marital relation has actually broken down. The assistance target person 40 is preferably targeted for a person of so-called working poor whose income is lower than an average income of a child-rearing household (e.g., equal to or less than a predetermined percentage), or a person whose income is unstable (e.g., a monthly income difference is more than a criterion value).

The assistance target person 40 is preferably targeted for a person who is not targeted for childcare assistance (subsidy payment or the like) of a government or the like or a person in a state of having difficulty raising a cost for taking advantage of a service of the same kind provided by a private sector.

Since the assistance target person 40 is a woman, the user 20 who assists the assistance target person 40 is preferably a woman. Moreover, since a woman has a higher risk of osteoporosis development and dementia development than a man, the user 20 is preferably targeted for a woman from a viewpoint of pre-symptomatic disease prevention. Thus, hereinafter, the user is also referred to as an “elderly woman.”

The figure illustrates one person as the user 20, one person as the assistance target person 40, and two persons as the children 42 of the assistance target person 40. However, a plurality of the users 20 and a plurality of the assistance target persons 40 can utilize the present work assisting system 1. Moreover, the number of the children 42 of the assistance target person 40 is not limited to two, but may be at least one, or equal to or more than two. In the work assisting system 1, a plurality of the users 20 and a plurality of the assistance target persons 40 are matched, and the user 20 assists the assistance target person 40 who is rearing a child.

The user terminal 30 and the assistance target person terminal 50 are computers such as smartphones, tablet terminals, and personal computers.

When utilizing the work assisting system 1, the user 20 and the assistance target person terminal 50 previously perform utilization registration. During utilization registration, for example, account information including an account name and a password for login authentication is registered. Alternatively, it may be coordinated with account information of an existing social networking service (SNS).

Further, identity verification may be performed at utilization, and biometric authentication information therefor may be previously registered, and login may be able to be performed by using the biometric authentication information. The biometric authentication information includes, for example, a feature value of at least one of a face, an iris, a vein, an auricle, a fingerprint, and the like. Further, the biometric authentication information may include biological information for behavioral biological authentication such as handwriting authentication, gait authentication, and keystroke authentication.

For a utilization method of a service of the present work assisting system 1, for example, method such as installing and starting a predetermined application on each terminal, or accessing a predetermined website from each terminal by using a browser or the like can be conceived. When the work assisting system 1 is logged into by using previously registered account information and authentication is successful, utilization of the service becomes possible.

### <Hardware Configuration Example>

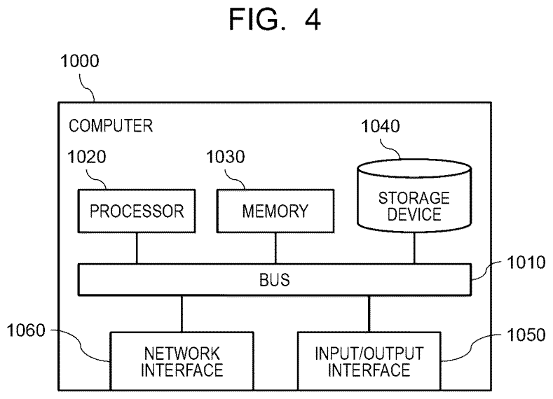

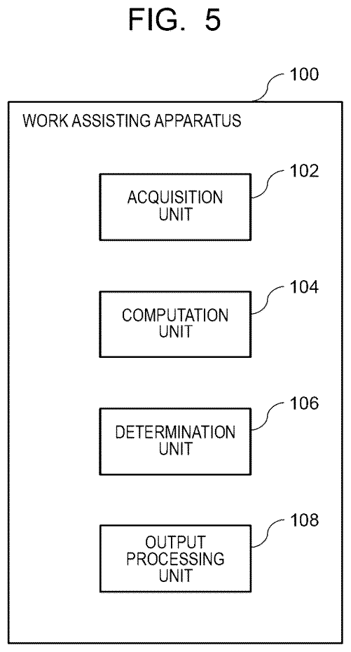

FIG. 4 is a block diagram illustrating a hardware configuration of a computer 1000 that achieves the work assisting apparatus 100 in FIG. 1 and FIGS. 5, 16, and 21 described later. The user terminal 30, the wearable terminal 32, and the assistance target person terminal 50 in FIG. 3 are also achieved by the computer 1000. Moreover, for functions of the work assisting apparatus 100, the user terminal 30 or the assistance target person terminal 50 may share and achieve some of the functions.

The computer 1000 includes a bus 1010, a processor 1020, a memory 1030, a storage device 1040, an input/output interface 1050, and a network interface 1060.

The bus 1010 is a data transmission path through which the processor 1020, the memory 1030, the storage device 1040, the input/output interface 1050, and the network interface 1060 transmit/receive data to/from one another. However, a method of mutually connecting the processor 1020 and the like is not limited to bus connection.

The processor 1020 is a processor achieved by a central processing unit (CPU), a graphics processing unit (GPU), or the like.

The memory 1030 is a main storage apparatus achieved by a random access memory (RAM) or the like.

The storage device 1040 is an auxiliary storage apparatus achieved by a hard disk drive (HDD), a solid state drive (SSD), a memory card, a read only memory (ROM), or the like. The storage device 1040 stores a program module that achieves each function (e.g., an acquisition unit 102, a computation unit 104, a determination unit 106, an output processing unit 108 that are described later, and a generation unit 110, a matching unit 112, and the like that are described later) of the work assisting apparatus 100. The processor 1020 reads each of the program modules onto the memory 1030, executes the read program module, and thereby achieves each function being relevant to the program module. Moreover, the storage device 1040 may also store each piece of data in the storage apparatus 120 of the work assisting apparatus 100.

The program module may be stored on a storage medium. A storage medium that stores the program module includes a non-transitory tangible medium usable by the computer 1000, and a program code readable by the computer 1000 (processor 1020) may be embedded in the medium.

The input/output interface 1050 is an interface for connecting the computer 1000 and various pieces of input/output equipment. The input/output interface 1050 also functions as a communication interface that performs near-field wireless communication such as Bluetooth (registered trademark) and near field communication (NFC).

The network interface 1060 is an interface for connecting the computer 1000 to a communication network. The communication network is, for example, a local area network (LAN) or a wide area network (WAN). A method of connecting the network interface 1060 to the communication network may be wireless connection or wired connection.

Then, the computer 1000 connects to necessary equipment (e.g., a display, an operation key, a touch panel, a camera, a speaker, and a microphone of the user terminal 30 or the assistance target person terminal 50, a display, a keyboard, a mouse, a speaker, and a microphone of the work assisting apparatus 100, and the like) via the input/output interface 1050 or the network interface 1060.

Each component of the work assisting apparatus 100 according to each example embodiment in FIG. 1 and FIGS. 5, 16, and 21 described later is achieved by any combination of hardware and software of the computer 1000 in FIG. 4. Then, it is appreciated by a person skilled in the art that there are a variety of modified examples of methods and apparatuses for the achievement. A functional block diagram illustrating the work assisting apparatus 100 according to each example embodiment illustrates not a configuration on a hardware basis but a block on a logical function basis.

### <Functional Configuration Example>

A functional configuration example of the work assisting apparatus 100 according to the example embodiment is described below.

FIG. 5 is a functional block diagram illustrating the functional configuration example of the work assisting apparatus 100 according to the example embodiment.

In addition to the configuration of the work assisting apparatus 100 in FIG. 1, the work assisting apparatus 100 further includes the output processing unit 108. The output processing unit 108 outputs, to an output apparatus (e.g., a display of the user terminal 30), the candidate of the task item determined by the determination unit 106.

Each component is described in detail below:

The acquisition unit 102 acquires biological information of the user 20, and resume information used when the user 20 applies for a job. Information acquired by the acquisition unit 102 is stored in the storage apparatus 120 in association with a user ID of the user 20.

The child 42 of the assistance target person 40 preferably includes a preschool child. A reason for this is that financial support from a government is increased for a school-aged child receives compared to a preschool child, and since a child can be left at school during daylight as compulsory education, it becomes easy to secure a work time of the assistance target person 40. Thus, the work assisting system 1 is intended to support particularly a mother with a preschool child between ages of 0 and 6 for whom governmental assistance is difficult to reach.

A job for which the user 20 assists the assistance target person 40 is, for example, at least one of housework and child rearing. For example, a job includes a housework task such as laundry, hanging out laundry, intake of laundry, a laundry folding task, cleaning, tidying up, garbage disposal, dish washing, and preparation of a meal that are performed at a residence of assistance target person 40, and, as taking care of the child 42, a child rearing task (so-called, taking care of a child) such as reading a picture book, playing together for a make-believe play and playing house, playing with a hand, singing (a nursery rhyme, a traditional children's song, or the like), supervising in block or brick playing, and helping with drawing and craft making.

Biological information acquired by the acquisition unit 102 includes health information indicating a health state of the user 20. Further, biological information including health information indicating a health state includes information relating to bone mass.

Since the acquisition unit 102 acquires health information indicating a health state of the user 20, and the computation unit 104 computes an evaluation value indicating a health risk, it becomes possible to evaluate a risk of development of dementia and osteoporosis before a stage of reaching disease, and it becomes possible to prevent a pre-symptomatic disease. Particularly, acquiring information relating to bone mass heightens the possibility that a development risk of osteoporosis that is unique to an elderly woman can be reduced preventively.

For example, a development risk of osteoporosis of the user 20 can be reduced by performing exercise assistance in which an assistance task to the assistance target person 40 is selected in such a way that the user 20 can acquire a load that improves an amount of daily activity. Alternatively, although described in detail in an example embodiment described below; a development risk of dementia of the user 20 can be reduced by performing interactive communication assistance which a task that particularly enables communication having interactivity by a conversation with the child 42 or the like is selected through an assistance task for the assistance target person 40.

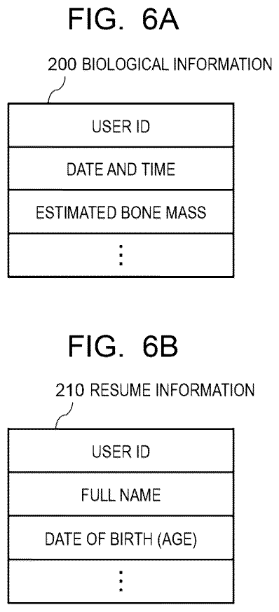

FIG. 6 is a diagram illustrating a data structure example of biological information 200 and resume information 210 of the user 20.

The biological information 200 of the user 20 may include at least one of pieces of health information indicating a health state of the user 20, such as weight, muscle mass, a body fat percentage, a visceral fat level, a subcutaneous fat percentage, a basal metabolic rate, a skeletal muscle percentage, a body mass index (BMI), a body age, a body water content, and estimated bone mass of the user 20 that are measured by, for example, a general body composition monitor.

In an example of FIG. 6 (a), the biological information 200, the biological information 200 of the user 20 includes at least identification information (user ID) of the user 20, an acquisition date and time of biological information, and information relating to bone mass (e.g., estimated bone mass). An acquisition date and time is preferably a date and time when biological information is measured. Moreover, when a plurality of types of biological information are acquired, it is preferable to include a measurement date and time of each piece of biological information.

In another example, the biological information 200 may include biological information acquired from the wearable terminal 32 worn by the user 20. For example, the biological information 200 may include at least one of a heart rate, the number of steps, calorie consumption, a walking record, an oxygen saturation, blood pressure, a body temperature, and electrocardiogram information. In the example of FIG. 6 (a), the acquisition date and time is preferably a date and time when biological information is measured by the wearable terminal 32, but is not limited thereto, and may be, for example, a date and time when the user terminal 30 receives the biological information from the wearable terminal 32.

Further, in another example, a user may input a measurement result of biological information from an input screen by using the user terminal 30, based on a test result at medical examination, a result of a health checkup, and the like.

In an example of FIG. 6 (b), the resume information 210 includes at least identification information (user ID), a name, and a date of birth (or an age) of the user 20. Moreover, the resume information 210 may further include a date and time when the information is acquired or stored.

The resume information 210 of the user 20 is information of a resume used when the user 20 applies for a job of assisting the assistance target person 40. The resume information 210 may include information of a general resume, for example, information relating to a name, a face photograph, a date of birth, an age, a gender, a family composition, an address, a work history, an educational background, a license, a special skill, and the like.

Resume information of the user 20 can be acquired, for example, by selecting an input screen of resume information from a menu screen (not illustrated) that is displayed when the present work assisting system 1 is logged in by using the user terminal 30, and accepting information input to the input screen by the user 20. A face photograph can be acquired by uploading image file data.

Further, resume information of the user 20 may include information relating to an application motivation of the user 20. For example, information indicating a degree of interest in improving person's own health, a degree of interest in interacting with another person, a degree of interest in contributing to society, and the like may be accepted by providing an input screen with an option previously or a free text input field.

Based on the application motivation, when health improvement is an application motivation, it also becomes possible for the determination unit 106 to change, based on the information, a task content to present to the user 20 as a candidate, into one with a high load in stages.

Each timing at which the acquisition unit 102 acquires the biological information 200 and the resume information 210 is different. The biological information 200 is acquired before utilization start of a service provided by the work assisting system 1. Further, thereafter, the biological information 200 may be acquired periodically for each type of biological information, for example, daily, monthly, semiannually, every other year, or the like, for a purpose of monitoring a health state of the user 20. The resume information 210 is acquired when applying for a job provided by the work assisting system 1. Further, the resume information 210 is preferably acquired when a change is made in content.

The computation unit 104 computes an evaluation value indicating a health risk for the user 20, by using the biological information 200 and the resume information 210 that have been acquired. The biological information 200 may include information relating to bone mass of the user 20, and an evaluation value indicating a health risk may include an evaluation value indicating a development risk of osteoporosis. An evaluation value may be, for example, a value indicating presence or absence of a risk of developing (e.g., 1 for “risk of developing present”, and 0 for “risk of developing absent”), or a value indicating a probability of developing.

Since a development risk of osteoporosis that is particularly a problem unique to an elderly woman can be evaluated, a possibility that a development risk of osteoporosis can be reduced preventively heightens.

The computation unit 104 computes an evaluation value indicating a development risk of osteoporosis, by using an estimated age of the user 20 estimated by using information relating to bone mass, and age information of the user 20 included in the resume information 210.

Specifically, the computation unit 104 acquires an age being associated with bone mass of the user 20 in the biological information 200 acquired by the acquisition unit 102, by using a table associating bone mass with age, and determines the acquired age as an estimated age. Then, the acquisition unit 102 acquires information relating to an age of the user 20 (e.g., a date of birth or an age) from the resume information 210, and the computation unit 104 computes an actual age of the user 20 from the acquired information relating to the age of the user 20.

The computation unit 104 compares the determined actual age with the estimated age, and, when the estimated age is older than an actual age by equal to or more than a certain age (e.g., 5 years older), determines an evaluation value indicating a health risk, as a value indicating that there is a development risk of osteoporosis.

However, an age difference between an actual age and an estimated age may change according to the actual age. For example, as an actual age is older, an age difference to be a criterion used for determination that there is a development risk of osteoporosis may be smaller. A reason for this is that, as an actual age becomes older, a velocity of decline in bone mass increases, and, therefore, an age difference in a percentage of decline in bone mass becomes smaller.

Moreover, as another example, when bone mass of the user 20 is included outside a range of a reference deviation a of distribution of reference bone mass with respect to an actual age, the computation unit 104 may determine an evaluation value of a health risk as a value (e.g., 1) indicating that there is a development risk. However, when bone mass is larger than the reference bone mass, the computation unit 104 determines an evaluation value of a health risk as a value (e.g., 0) indicating that there is no development risk of osteoporosis.

Moreover, as yet another example, by using a table in which information of average bone mass by age is registered, the computation unit 104 may compare the estimated bone mass of the user 20 in the biological information 200 acquired by the acquisition unit 102 with an average value of bone mass at the same age as the user 20, and, when the estimated bone mass of the user 20 is smaller than the average value by equal to or more than a predetermined value, an evaluation value of a health risk may be determined as a value indicating that there is a development risk of osteoporosis.

Note that, in a computation process of an osteoporosis development risk by the computation unit 104, when it can be assessed that the estimated bone mass of the user 20 is within a range of “development” (indicating an abnormal value), the output processing unit 108 preferably outputs notification information recommending having a medical examination (thorough examination) at a hospital or the like before the assignment of a task by the work assisting system 1.

In this way, a development risk of osteoporosis that is particularly a problem unique to an elderly woman can be evaluated, a possibility that a development risk of osteoporosis can be reduced preventively heightens.

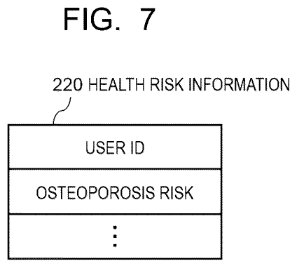

FIG. 7 is a diagram illustrating a data structure example of health risk information 220. The health risk information 220 includes, in association with a user ID, an evaluation value indicating a development risk of osteoporosis computed by the computation unit 104. The evaluation value indicating the development risk of osteoporosis is stored in the storage apparatus 120 as the health risk information 220 in association with the user ID of the user 20.

The determination unit 106 determines, by using an evaluation value indicating a health risk, a candidate of a task that the user 20 is capable of taking charge of among jobs.

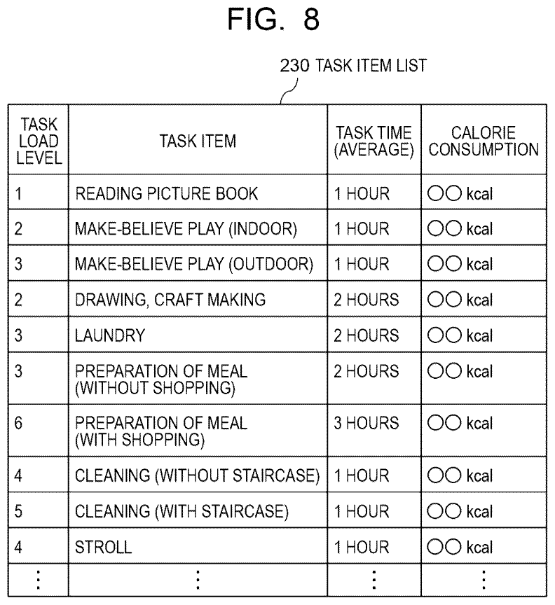

FIG. 8 is a diagram illustrating a data structure example of a task item list 230. The task item list 230 includes, for each task item, a task load level, an assumed task time, and a value of an average calorie consumption when the task is performed for a task time. The task item list 230 is stored in the storage apparatus 120.

In this example, a task load level is set at 1 to 6 stages in a range of calorie consumption in ascending order of calorie consumption, i.e., in ascending order of load. However, the task load level may be at a plurality of stages, and is not limited to six stages.

Moreover, the task item list 230 may be generated for each assistance target person. This is because a task load may differ even for the same task content according to status of the assistance target person 40.

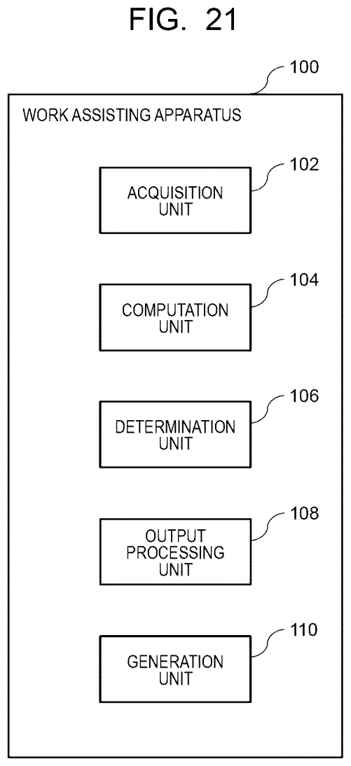

FIG. 21 is a functional block diagram illustrating another functional configuration example of the work assisting apparatus 100 according to the example embodiment.

The acquisition unit 102 acquires target person attribute information indicating an attribute of the assistance target person 40.

The work assisting apparatus 100 may further include a generation unit 110 that generates the task item list 230, based on the target person attribute information.

The determination unit 106 determines a candidate of a task to present to the user 20 from among tasks included in the task item list 230 generated by the generation unit 110.

The task item list 230 includes load information for each task item for each of the assistance target persons 40.

The target person attribute information may further include attribute information of the child 42, such as the number of the children 42 of the assistance target person 40, a gender, and an age.

For example, load information (e.g., calorie consumption) in the task item list 230 may differ according to the number of the children 42 of the assistance target person 40, a gender, an age, and the like.

Since a task of the user 20 includes taking care of the child 42 of the assistance target person 40, a case can be conceived where a load of the task differs according to an age, a gender, and the number of the children 42. Thus, since the task item list 230 in which a task load considering an attribute of the child 42 is set in addition to the assistance target person 40 can be used for determination of a task candidate by the determination unit 106, a task load of a task to present to the user 20 can be accurately determined, and it becomes possible to present a task with an appropriate task load to the user 20.

The determination unit 106 determines an allowable load range of the user 20, based on an evaluation value indicating a health risk, and determines, based on load information determined for each task, a candidate of a task to present to the user 20.

For example, the determination unit 106 may determine a record of exercise habit by using a measurement result of an activity meter of the user 20, and determine a range of an allowable load of the user 20.

Note that, an allowable load range may be a restriction on a task load of each task, or may be a restriction on a total task load when the user 20 selects a plurality of tasks. In the latter case, it may be configured such that the output processing unit 108 totals a task load of a task selected by the user 20 in a task candidate screen 300 presented to the user 20, and, when the allowable load range is exceeded, a message notifying the fact may be output, or a task exceeding the allowable load range cannot be selected. Moreover, the output processing unit 108 may propose an alternative task satisfying the allowable load range.

The total of a task load of the task described above may be a total of values of the task load levels described above, or may be a total of calorie consumptions indicating a task load of each task. Moreover, a task load may be set by using, for example, a task environment condition described later. For example, in a case of a task in a residence with a staircase, in order to add up a calorie consumption resulting from going up and down the staircase, a calorie consumption acquired by multiplying a total of calorie consumptions of a task needing going up and down the staircase or the whole task by a predetermined coefficient (a value larger than 1) may be used, or a task load level may be enhanced by a predetermined stage.

In this way, since a range of an allowable load considering a record of an exercise habit of the user 20 can be set, it becomes possible for the user 20 having an exercise habit to select a task with a higher load, and the user 20 having no exercise habit can expect an effect of preventing an accident and an injury during a task by avoiding selection of a task with a load being too high.

Specifically; the determination unit 106 acquires an evaluation value of a health risk for the user 20 from, for example, the health risk information 220 in FIG. 7, and, when the evaluation value of a health risk satisfies a criterion, the user 20 determines, based on the evaluation value of the health risk, a task load level in a range of an allowable load, extracts a task item being relevant to the determined task load level from the task item list 230 in FIG. 8, and determines the task item as a candidate of a task that the user 20 is capable of taking charge of. Herein, a criterion includes a matter that an evaluation value of a health risk indicates that a lightening of a task load is necessary or a task load needs to be increased.

When an evaluation value of a health risk does not satisfy the criterion, i.e., when an evaluation value indicates that the user 20 has no health risk, and, therefore, there is no need for lightening or increase or decrease of a task load, the determination unit 106 may not perform determination of a task load level of the user 20, and may determine all task items as candidates of tasks that the user 20 is capable of taking charge of.

When an evaluation value of a health risk satisfies a criterion, i.e., when an evaluation value indicates that the user 20 has a health risk, and, therefore, lightening or lightening or increase or decrease of a task load is necessary, the determination unit 106 puts a restriction on a task load level set for each task item, extracts a task item that does not exceed the restriction, and determines the task item as a task candidate for the user 20. For example, when an evaluation value of a health risk for the user 20 indicates that there is a development risk of osteoporosis, a restriction of a task load level is set to a first criterion value (e.g., level 4 of a multistage level), and a task whose task load level is equal to or less than the first criterion value is extracted and set as a candidate. Alternatively, for example, the determination unit 106 may extract a task whose task load level in the task item list 230 is equal to or more than a second criterion value (e.g., level 5 of the multistage level), and determine the extracted task as a candidate. Moreover, when a health risk indicates a development risk of dementia described later, determination may be performed by using another criterion value.

In this way, even in the same case where there is a development risk of osteoporosis, a restriction method of a task load level may differ. For example, it can be caused to differ according to a daily exercise habit of the user 20, a basic physical strength, and absence or presence of a health improvement desire of the user 20. In this way, a task can be selected according to a load tolerance range of the user 20. Note that, presence or absence of a health improvement desire of the user 20 can be assessed by referring to information relating to an application motivation in the resume information described above.

Moreover, when there is no daily exercise habit of the user 20, it is preferable to initially set a task load level low. It is expected first of all that an opportunity for an exercise habit is provided by a low-load task, and establishment of the habit can be promoted. Thereafter, when physical strength of the user 20 improves by continuing work and establishing an exercise habit, and an evaluation value reaches a stage where improvement of a health risk appears, the determination unit 106 may enhance the task load level, based on the evaluation value. In this way, the user 20 who does not have a daily exercise habit can also expect that establishment of an exercise habit reduces a development risk of osteoporosis and can contribute to improvement of healthy life expectancy.

### <Operation Example>

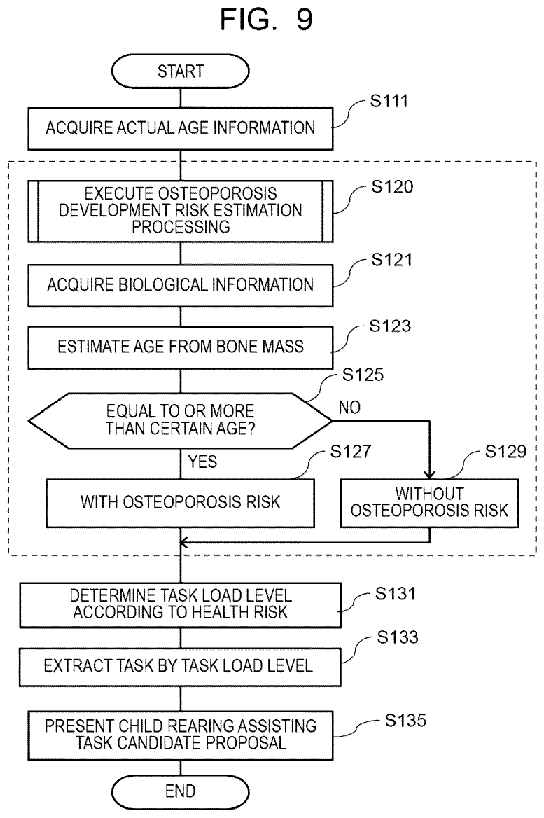

FIG. 9 is a flowchart illustrating an operation example of the work assisting apparatus 100 according to the example embodiment.

This flow is executed first time the user 20 starts utilization of a service of the work assisting system 1.

Note that, steps S111 and S121 in FIG. 9 correspond to step S101 in FIG. 2, and steps S123 to S129 in FIG. 9 correspond to step S103 in FIG. 2. Steps S131 to S135 in FIG. 9 correspond to step S105 in FIG. 2.

First, the acquisition unit 102 acquires the actual age information of the user 20 from the resume information 210 (step S111). Then, the computation unit 104 executes a processing routine (S120) of estimating a development risk of osteoporosis. The osteoporosis development risk estimation processing routine S120 includes steps S121 to S129 surrounded by a broken line in the figure.

In the osteoporosis development risk estimation processing routine S120, the computation unit 104 first acquires the biological information 200 (step S121). Herein, bone mass of the user 20 is acquired as the biological information 200. Then, the computation unit 104 computes an age estimated from the bone mass of the user 20, and determines the computed age as an estimated age (step S123).

Then, the computation unit 104 compares the actual age acquired in step S121 with the estimated age estimated in step S123, and, when the estimated age is older than the actual age by equal to or more than a certain age (YES in step S125), determines the evaluation value of the development risk of osteoporosis to be with-development-risk, as an evaluation value of a health risk for the user 20 (e.g., 1) (step S127). On the other hand, when the estimated age is not older than the actual age by equal to or more than a certain age (NO in step S125), the computation unit 104 determines the evaluation value of the development risk of osteoporosis to be without-development-risk, as an evaluation value of the health risk for the user 20 (e.g., 0) (step S129). The evaluation value of the development risk of osteoporosis is stored in the health risk information 220 (FIG. 7) in association with the user ID of the user 20 as a health risk evaluation value.

Then, the determination unit 106 acquires an evaluation value of a health risk for the user 20 from the health risk information 220, and determines a task load level according to the evaluation value of the health risk for the user 20 (step S131). For example, when there is a development risk of osteoporosis, the determination unit 106 may determine a task load level in the task item list 230 to be equal to or less than 4 according to the evaluation value of the development risk of osteoporosis. In this case, a task with a task load level of equal to or less than 4 in the task item list 230 is extracted as a candidate (step S133). Conversely, when there is a development risk of osteoporosis, the determination unit 106 may determine a task load level in the task item list 230 to be equal to or more than 3 according to an evaluation value of the development risk of osteoporosis. In this case, a task with a task load level of equal to or more than 3 in the task item list 230 is extracted as a candidate (step S133).

Herein, when an evaluation value of a development risk of osteoporosis is a value indicating that the development risk of osteoporosis is higher than a criterion, a task with a high task load level is preferably set to a low level at start in order to avoid a possibility of involving a danger such as a stress fracture. Thereafter, when the evaluation value of the development risk becomes lower than the criterion, it is preferable to set the task load level gradually high.

On the other hand, when an evaluation value of a development risk of osteoporosis indicates that there is a development risk, but is lower than a criterion, a task load level may be set slightly high with an aim of health improvement by performing a task. In this case as well, it is preferable to set to a low level at start of work, and, when there is no problem after monitoring an evaluation value of a health risk for the user 20 for a predetermined period, it is preferable to gradually set setting high.

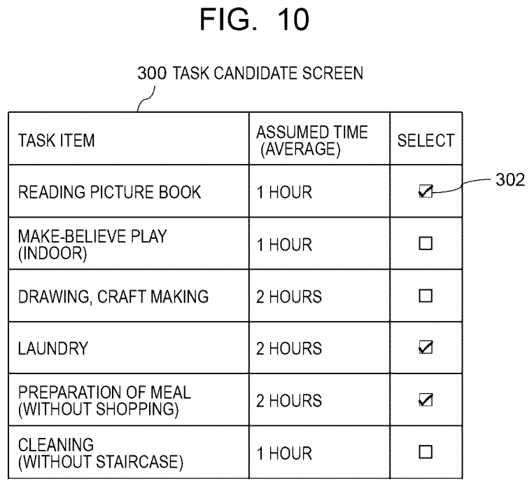

The output processing unit 108 presents the task extracted in step S133 to the user 20 as a candidate of a task that the user 20 is capable of taking charge of (step S135). For example, the output processing unit 108 screen-displays the task candidate screen 300 in FIG. 10 on the display of the user terminal 30.

FIG. 10 is a diagram illustrating one example of the task candidate screen 300. The task candidate screen 300 includes a list of candidates of tasks that the user 20 is capable of taking charge of. The list on the task candidate screen 300 includes a task item, a time assumed to be taken for the task, and a check box 302 that accepts selection of a task that the user 20 desires to take charge of from among candidates.

In the task candidate screen 300 (FIG. 10), the user 20 can select, from among assigned tasks, a task item that the user 20 desires to take charge of.

As described above, according to the present example embodiment, the work assisting apparatus 100 includes the acquisition unit 102, the computation unit 104, the determination unit 106, and the output processing unit 108. The acquisition unit 102 acquires biological information of the user 20 and resume information of the user 20. The computation unit 104 computes an evaluation value indicating a health risk for the user 20, by using the biological information and the resume information that have been acquired. The determination unit 106 determines, by using the evaluation value indicating the health risk, a candidate of a task to present to the user 20. The output processing unit 108 outputs, to an output apparatus (e.g., the display of the user terminal 30), the candidate of the task item determined by the determination unit 106.

According to this configuration, taking a job suited to a health state can be appropriately assisted in a simple process. Particularly, from a perspective of a pre-symptomatic disease, work targeting an elderly person at a stage of being in a health state that does not require diagnosis at a medical institution or the like, particularly, a woman who is at high development risk of dementia and osteoporosis can be appropriately assisted. This is expected to enable extension of healthy life expectancy; and help solve a social problem such as an increase in medical cost and nursing care cost.

Furthermore, it is possible to give an elderly person (particularly am elderly woman) an opportunity for social participation and provide a point of contact with society, while considering safety in work. Further, taking advantage of an elderly human resource becomes possible. For example, in taking advantage of an elderly human resource, taking advantage of a highly specialized and qualified person and a human resource with long work experience is expected. The work assisting system 1 similarly enables to provide an opportunity for social participation to an elderly human resource with no work experience by focusing on a length of housewife experience of an elderly woman and highness of experience.

Moreover, since the assistance target person 40 is a single mother or the like with a preschool child, a single mother with a preschool child who is not an elementary or junior high school child receiving schooling assistance (requiring learning assistance) can be assisted. Since a task of the user 20 includes taking care of the child 42 who is a preschool child, stable continuation of work of a single mother is also assisted, and it also becomes possible to encourage social participation and financial independence of a single mother. Thus, there is a possibility of becoming a clue to solution of a poverty problem of a woman.

Moreover, since a task load level can be restricted and a candidate of a task can be determined, assignment of a reasonable task considering health of an elderly woman at work becomes possible. Moreover, since a development risk of osteoporosis that is a problem particularly unique to an elderly woman can be evaluated, and a task based on an evaluation value can be determined as a candidate, a possibility that the development risk of osteoporosis can be reduced is also high.

By performing a task, the user 20 can improve a daily activity level, and it becomes possible to maintain or improve a health state.

### Second Example Embodiment

The present example embodiment is similar to the first example embodiment described above, except for having a configuration in which a task candidate is determined by using a development risk of dementia as an evaluation value of a health risk. Since a work assisting apparatus 100 according to the present example embodiment has the same configuration as the first example embodiment, description is given by using FIG. 5. Moreover, the configuration according to the present example embodiment may be combined with at least one of configurations according to other example embodiments to an extent that no contradiction occurs.

### <Functional Configuration Example>

In the present example embodiment, biological information 200 acquired by an acquisition unit 102 includes a face image of a user 20.

A computation unit 104 computes an evaluation value indicating a health risk, based on an age of the user 20 estimated from the face image and an age of the user 20 included in resume information 210.

As a health risk computed from a face image, for example, a development risk of dementia, a development risk of arteriosclerosis, and the like can be conceived. A development risk of arteriosclerosis is described in another example embodiment described later.

A development risk of dementia is described below.

Specifically, the acquisition unit 102 acquires, for example, a face image of the user 20 included in the resume information 210 of the user 20. Alternatively, the acquisition unit 102 may acquire a video captured during an online interview with the user 20. The computation unit 104 causes an image processing apparatus (not illustrated) to perform age estimation processing by using the face image of the user 20. The age estimation technique can use a general technique, and is not particularly limited. Further, the acquisition unit 102 acquires information (e.g., a date of birth or an age) relating to an age of the user 20 from the resume information 210. Then, the computation unit 104 determines an actual age of the user 20 from information relating to the age of the user 20 acquired by the acquisition unit 102.

Then, the computation unit 104 compares the determined actual age with the estimated age, and, when the estimated age is older than the actual age by equal to or more than a certain age (e.g., 5 years older), determines an evaluation value indicating a health risk, as a value indicating that there is a development risk of dementia.

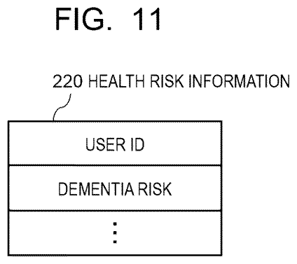

FIG. 11 is a diagram illustrating a data structure example of health risk information 220. The health risk information 220 includes, in association with a user ID, an evaluation value indicating a development risk of dementia computed by the computation unit 104. The evaluation value indicating the development risk of dementia is stored in a storage apparatus 120 as the health risk information 220 in association with the user ID of the user 20.

The determination unit 106 determines, by using an evaluation value indicating a health risk, a candidate of a task that the user 20 is capable of taking charge of among jobs.

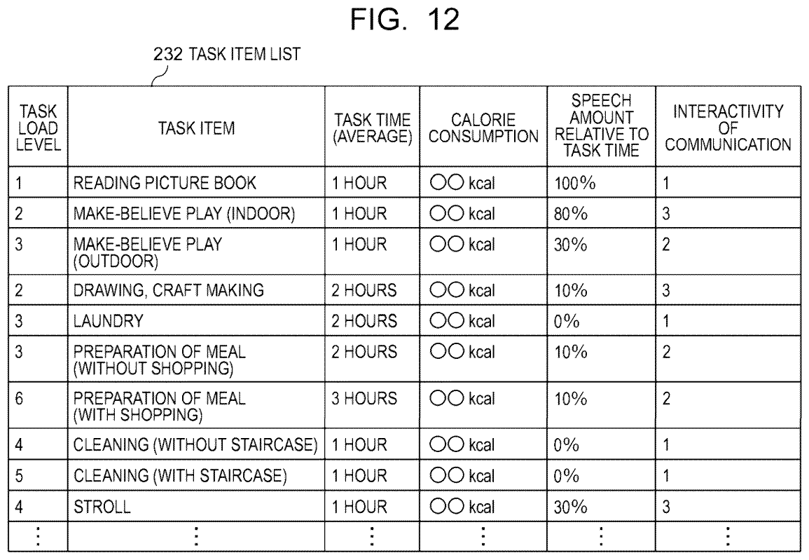

FIG. 12 is a diagram illustrating a data structure example of a task item list 232.

A task item list 230 in FIG. 8 is for exercise assistance required, and the task item list 232 in FIG. 12 is for communication assistance required. The task item list 232 is provided separately from the task item list 230 for exercise assistance required in FIG. 8, and stored in the storage apparatus 120.

For each task item, the task item list 232 stores, in relation to each other, an average value of a task time assumed to be taken for the task, a calorie consumption per unit time when the task is performed, percentage (%) of a speech amount to a task time assumed during the task, and an interactivity level of communication in a task. The percentage of a speech amount is an index indicating a degree of continuity of conversation (length of exchange).

An interactivity level of communication is set to the following three stages in this example, but is not limited thereto.

Level 3 expects continuous conversational exchange.

Level 2 is a level where there is conversational exchange, but there is a possibility of ending up with a single exchange.

Level 1 is a level where there is a possibility of ending up with one-sided talking, and is, for example, a level where reading a picture book is possible.

Herein, an interactivity level of communication of a task is divided into three stages of levels 1 to 3. The interactivity of communication is a level indicating how much exchange of conversation there is, and indicates that an amount of conversation is larger as the level becomes the levels 1 to 3.

For example, the determination unit 106 acquires an evaluation value of a health risk for the user 20 (in the present example embodiment, an evaluation value of a development risk of dementia) from the health risk information 220 in FIG. 11, extracts a task item with interactivity of communication at the level 3 from the task item list 232 when the evaluation value of the health risk satisfies a criterion, i.e., indicates that there is a development risk of dementia (communication assistance required), and determines the task item as a candidate of a task that the user 20 is capable of taking charge of.

When the evaluation value of the health risk does not satisfy the criterion, i.e., when the evaluation value indicates that the user 20 has no health risk and therefore communication assistance is not needed, the determination unit 106 may include all task items as candidates of tasks that the user 20 is capable of taking charge of. However, some task items may be included and determined as candidates.

For example, when an evaluation value of a health risk for the user 20 indicates that there is a development risk of dementia, the determination unit 106 restricts a level of interactivity of communication to the level 3, and extracts only a task of the level 3. Alternatively, a task may be extracted by using a speech amount in the task item list 232. For example, the determination unit 106 may extract a task with a speech amount equal to or more than a predetermined value.

For example, a speech amount may be measured by capturing a scene where the user 20 is actually performing a task of contacting a child 42 of an assistance target person 40, and performing analysis processing of generated video data. The determination unit 106 may determine a task by using the speech amount. Moreover, a task may be determined by assuming a case where, although a level of interactivity of communication of the user 20 is low; the child 42 of assistance target person 40 is sociable and has a large speech amount, and, therefore, the user 20 feels tired. For example, when a level of interactivity of communication of the user 20 is low, or when the child 42 of the assistance target person 40 is sociable and has a large speech amount, the determination unit 106 may include, in a candidate, a task with a low level of interactivity of communication.

### <Operation Example>

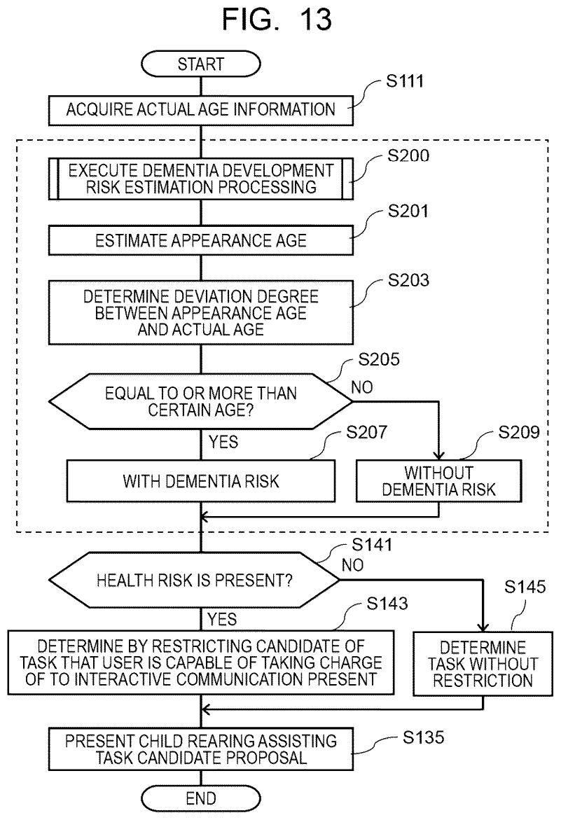

FIG. 13 is a flowchart illustrating an operation example of the work assisting apparatus 100 according to the example embodiment.

This flow is executed first time the user 20 starts utilization of a service of a work assisting system 1. Moreover, the flow in FIG. 13 includes step S111 being the same as that in the flow in FIG. 9, and step S135. The flow in FIG. 13 is the same as the flow in FIG. 9 except for including a processing routine (S200) for estimating a development risk of dementia in place of the osteoporosis development risk estimation processing routine S120 in FIG. 9, and including steps S141 to S145 in place of steps S131 to S133 in FIG. 9.

First, the acquisition unit 102 acquires actual age information of the user 20 from the resume information 210 (step S111). Then, the computation unit 104 executes a dementia development risk estimation processing routine (S200). The dementia development risk estimation processing routine S200 includes steps S201 to S209 surrounded by a broken line in the figure.

In the dementia development risk estimation processing routine S200, the computation unit 104 first acquires the biological information 200 (step S201). Herein, a face image of the user 20 is acquired as the biological information 200. As the face image of the user 20, for example, the face image of the user 20 included in the resume information 210 is acquired. Then, the computation unit 104 causes the image processing apparatus to perform age estimation processing by using the face image of the user 20, and acquires an estimated age (step S203).

Then, the computation unit 104 compares the actual age acquired in step S201 with the estimated age acquired in step S203, and, when the estimated age is older than the actual age by equal to or more than a certain age (YES in step S205), determines the evaluation value of the development risk of dementia to be with-development-risk, as an evaluation value of a health risk for the user 20 (e.g., 1) (step S207). On the other hand, when the estimated age is not older than the actual age by equal to or more than a certain age (NO in step S205), the computation unit 104 determines the evaluation value of the development risk of dementia to be without-development-risk, as an evaluation value of the health risk for the user 20 (e.g., 0) (step S209). The evaluation value of the health risk is stored in the health risk information 220 (FIG. 11).

Then, the determination unit 106 refers to the health risk information 220, assesses presence or absence of a health risk for the user 20, and, when there is a health risk (YES in step S141), determines a candidate of a task that the user 20 is capable of taking charge of, which is restricted to a candidate of a task being relevant to the evaluation value of the health risk for the user 20 (step S143). For example, when there is a health risk (herein, a development risk of dementia), the determination unit 106 may restrict the interactivity of communication of the task item list 230 to the level 3. In this case, a task with a level of interactivity of task communication of 3 in the task item list 230 is extracted as a candidate.

When there is no health risk (NO in step S141), no restriction is placed on a task item, and the determination unit 106 determines all task items in the task item list 230 as candidates of tasks that the user 20 is capable of taking charge of (step S145). Note that, it is premised that the task item list 230 includes a task targeting an elderly woman. In other words, in consideration of standard physical strength or the like of an elderly woman, the task item list 230 preferably includes a task with a load suited to a health state of an elderly woman, and no task to be an overload for an elderly woman is preferably included.

The output processing unit 108 presents a task determined in step S143 or step S145 to the user 20 as a candidate of a task that the user 20 is capable of taking charge of (step S135). For example, the output processing unit 108 screen-displays the task candidate screen 300 in FIG. 10 on a display of the user terminal 30. In the task candidate screen 300, the user 20 can select, from among assigned tasks, a task item that the user 20 desires to take charge of.

As described above, according to the present example embodiment, the biological information 200 acquired by the acquisition unit 102 includes a face image of the user 20. Then, the computation unit 104 estimates an age of the user 20 from the face image. The computation unit 104 computes, by using a difference between an age acquired from an estimation result of an age and an actual age of the user 20 included in the resume information 210, an evaluation value indicating a development risk of dementia as an evaluation value indicating a health risk.

In this way, the work assisting apparatus 100 according to the present example embodiment not only provides an effect similar to the example embodiment described above, but also evaluates a development risk of dementia that is a problem particularly unique to elderly women, and can determine, as a candidate, a task based on an evaluation value, and, therefore, a possibility that a development risk of dementia can be reduced while assisting getting work of an elderly woman heightens.

### Third Example Embodiment

In the first example embodiment described above, a computation unit 104 estimates an age of a user 20 by using bone mass of the user 20 as biological information, and computes an evaluation value of a development risk of osteoporosis by using a difference between an actual age and an estimated age. On the other hand, in a second example embodiment, the computation unit 104 estimates an age of the user 20 by using a face image of the user 20 as biological information, and computes an evaluation value for a development risk of dementia by using the difference between the actual age and the estimated age. In the present example embodiment, the computation unit 104 estimates each of the ages by using both bone mass and a face image of the user 20 as biological information, and computes both an evaluation value of development risk of osteoporosis and an evaluation value of a development risk of dementia.

The work assisting apparatus 100 according to the present example embodiment has the same configuration as the first and second example embodiments, and is therefore described by using FIG. 5. However, a configuration according to the present example embodiment may be combined with at least one of configurations according to other example embodiments to the extent that no contradiction occurs.

### <Functional Configuration Example>

Biological information 200 acquired by an acquisition unit 102 includes information relating to bone mass of the user 20, and a face image. Resume information 210 acquired by the acquisition unit 102 includes information relating to an age of the user 20 (e.g., a date of birth or an age).

The computation unit 104 computes an evaluation value indicating a health risk for the user 20 by using the biological information 200 and the resume information 210 that have been acquired.

Specifically; the computation unit 104 determines an actual age of the user 20 from information relating to the age of the user 20 acquired by the acquisition unit 102. The computation unit 104 acquires an age being relevant to estimated bone mass of the user 20 by using a table of average bone mass by age, based on the estimated bone mass of the user 20 in the biological information 200 acquired by the acquisition unit 102 by using a table associating bone mass with an age, and determines the age as the estimated age. On the other hand, the computation unit 104 causes an image processing apparatus to perform age estimation processing by using a video of a face of the user 20, and acquires the estimated age of the user 20.

Then, the computation unit 104 compares the determined actual age with the estimated age based on the bone mass, and, when the estimated age is older than the actual age by equal to or more than a certain age (e.g., 5 years older), determines the evaluation value indicating a health risk to be a value indicating that there is a development risk of osteoporosis. Further, the computation unit 104 compares the determined actual age with the estimated age based on the face image, and, when the estimated age is older than the actual age by equal to or more than a certain age (e.g., 5 years older), determines an evaluation value indicating a health risk to be a value indicating that there is a development risk of dementia.

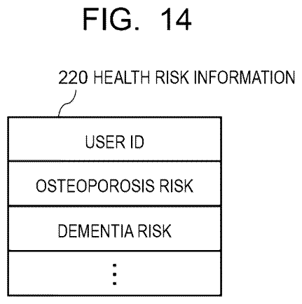

FIG. 14 is a diagram illustrating a data structure example of health risk information 220 according to the present example embodiment. The health risk information 220 includes, in association with a user ID, an evaluation value indicating a development risk of osteoporosis computed by the computation unit 104 and an evaluation value indicating a development risk of dementia. The evaluation value indicating a development risk of dementia of the user 20 and the evaluation value indicating a development risk of dementia of the user 20 are stored in the storage apparatus 120 as the health risk information 220 in association with the user ID of the user 20.

A determination unit 106 determines, by using an evaluation value indicating a health risk, a candidate of a task that the user 20 is capable of taking charge of among jobs.

In the example embodiment described above, a candidate of a task that the user 20 is capable of taking charge of is determined by using an evaluation value of one type of health risk. In the present example embodiment, a candidate of a task that the user 20 is capable of taking charge of is determined by using evaluation values indicating two types of health risks. A method of handling an evaluation value indicating the two types of health risks is exemplified below; but is not limited to thereto.
(a1) An evaluated value of an osteoporosis development risk and an evaluated value of a dementia development risk are added and divided by 2, and an evaluated value of a health risk is computed. For example, in addition to simple addition, a method of deriving by focusing on a relationship between a development risk of each disease and considering a correlation can also be conceived.
(a2) When adding in (a1), an evaluation value of each risk is multiplied by a coefficient, and an evaluation value of a health risk is computed. A sum of two coefficients is caused to be 1. Alternatively, for example, an evaluation value may be caused to be converged to 0 to 1 by machine learning, after taking into account a task environment condition.
(a3) When one of the evaluation values of development risks of osteoporosis and dementia indicates that there is a development risk, the evaluation value is used.
(a4) Each of load levels enabling a task that are determined by using evaluation values of development risks of osteoporosis and dementia is determined.

Then, the determination unit 106 acquires the evaluation value of the health risk for the user 20 determined as described above, and, when the evaluation value of the health risk satisfies a criterion, determines, based on the evaluation value of the health risk, a task load level that the user 20 is capable of taking charge of, extracts, from a task item list 230 in FIG. 8, a task item being relevant to the determined task load level, and determines the task item as a candidate of a task that the user 20 is capable of taking charge of. Herein, the criterion includes a matter that an evaluation value of a health risk indicates that lightening of a task load is necessary. Since the restriction on the task load level based on a health risk is the same as in the first example embodiment described above, description is not included.

However, in a case of (a4) described above, there are two evaluation values indicating a health risk. Thus, in a case of (a4), two types of the task item lists 230 may be prepared for osteoporosis and for dementia. The determination unit 106 may extract, from each of the relevant task item list 230, a task item being relevant to each of load levels being relevant to the two types of evaluation values determined in (a4).

### <Operation Example>

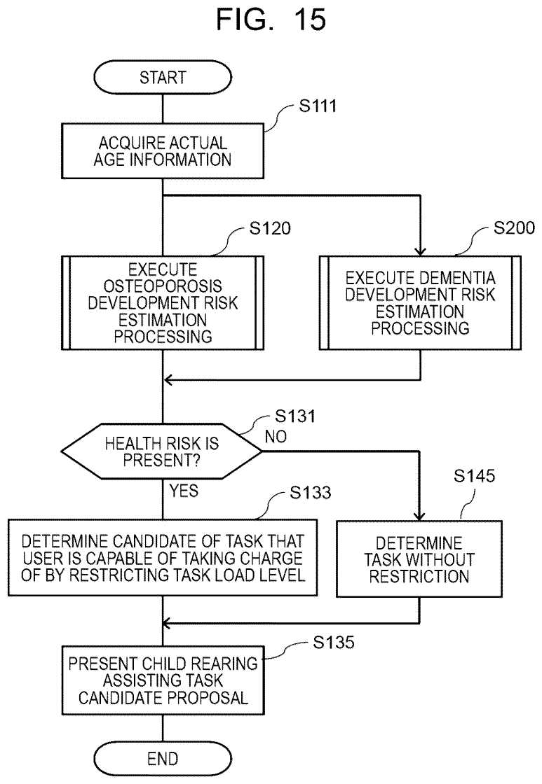

FIG. 15 is a flowchart illustrating an operation example of the work assisting apparatus 100 according to the example embodiment.

This flow is executed first time the user 20 starts utilization of a service of a work assisting system 1. Moreover, the flow in FIG. 15 includes step S111 being the same as that in the flow in FIG. 9, an osteoporosis development risk estimation processing routine S120, and steps S131 to S135, and further includes the dementia development risk estimation processing routine S200 in FIG. 13, and step S145.

First, the acquisition unit 102 acquires actual age information of the user 20 from the resume information 210 (step S111). Then, the computation unit 104 executes a processing routine (S120) of estimating a development risk of osteoporosis. This processing routine is the same as the processing described in the first example embodiment. By the processing routine S120, the computation unit 104 computes an evaluation value of a development risk of osteoporosis of the user 20. The computed evaluation value of the development risk of osteoporosis is stored in the storage apparatus 120 as the health risk information 220 (FIG. 14) in association with the user ID of the user 20.

Further, in parallel with the processing routine S120, the computation unit 104 executes the dementia development risk estimation processing routine (S200). By the processing routine S200, the computation unit 104 computes an evaluation value of a development risk of dementia of the user 20. However, the processing routine S120 and the processing routine S200 may not be parallel processing, and may be sequential processing. The computed evaluation value of the development risk of dementia is further stored in the storage apparatus 120 as the health risk information 220 (FIG. 14) in association with the user ID of the user 20.

Then, the determination unit 106 refers to the health risk information 220 (FIG. 14), assesses presence or absence of a health risk for the user 20, and, when there is a health risk (YES in step S131), determines a candidate of a task that the user 20 is capable of taking charge of, which is restricted to a candidate of a task being relevant to the evaluation value of the health risk for the user 20 (step S133). For example, when an evaluation value of at least one of a development risk of osteoporosis and a development risk of dementia indicates that there is a development risk of osteoporosis, the determination unit 106 may restrict a task load level in the task item list 230 to equal to or less than 4. In this case, a task with a task load level of equal to or less than 4 in the task item list 230 is extracted as a candidate.

When the evaluation values of the development risk of osteoporosis and the development risk of dementia both indicate that there is no development risk (NO in step S131), no restriction is placed on a task item, and the determination unit 106 determines all the task items in the task item list 230 as candidates of tasks that the user 20 is capable of taking charge of (step S145).

An output processing unit 108 presents the task determined in step S133 or step S145 to the user 20 as a candidate of a task that the user 20 is capable of taking charge of (step S135). For example, the output processing unit 108 screen-displays a task candidate screen 300 in FIG. 10 on a display of a user terminal 30. In the task candidate screen 300 (FIG. 10), the user 20 can select, from among assigned tasks, a task item that the user 20 desires to take charge of.

As described above, according to the present example embodiment, the computation unit 104 computes an evaluation value indicating a development risk of osteoporosis and an evaluation value indicating a development risk of dementia, and the determination unit 106 determines, by using the evaluation values, a candidate of a task that the user 20 is capable of taking charge of.

In this way, the work assisting apparatus 100 according to the present example embodiment can provide an effect similar to the example embodiment described above, evaluate both a development risk of osteoporosis and a development risk of dementia that are particularly problems unique to an elderly woman, and determine, as a candidate, a task based on both the evaluation values, and, therefore, a possibility that development risks of both osteoporosis and dementia can be reduced while assisting getting work of an elderly woman heightens.

Then, when development risks of osteoporosis and dementia can be reduced, it is expected to help solve a problem of rises in medical cost and nursing care cost due to an aging society, so-called a 2025 problem.

### Fourth Example Embodiment

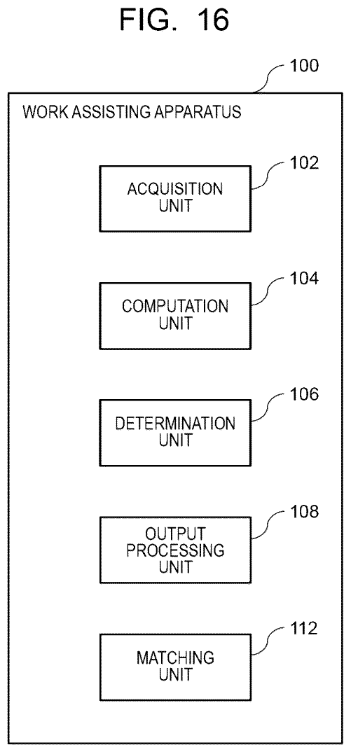

FIG. 16 is a functional block diagram illustrating a functional configuration example of a work assisting apparatus 100 according to an example embodiment.

The work assisting apparatus 100 according to the present example embodiment is similar to the first to third example embodiments described above, except for having a configuration that matches a user 20 and an assistance target person 40. In addition to the configuration of the work assisting apparatus 100 in FIG. 5, the work assisting apparatus 100 according to the present example embodiment further includes a matching unit 112. However, the configuration according to the present example embodiment may be combined with at least one of configurations according to other example embodiments to an extent that no contradiction occurs.

### <Functional Configuration Example>

A task includes a task relating to a child 42 of an assistance target person 40.

Resume information 210 includes information relating to the experience of child rearing of the user 20.

An acquisition unit 102 acquires assistance target person attribute information including an attribute of the child 42 of the assistance target person 40.

The matching unit 112 generates a combination of the user 20, the assistance target person 40, and the child 42 by using the resume information 210 and the assistance target person attribute information.

The resume information 210 includes presence or absence of child-rearing experience of the user 20, and a gender of the child. Further, the resume information 210 may include a family structure (a daily conversation amount) of the user 20.

Moreover, the resume information 210 may include information relating to “personality” of the child 42 of the user 20 as attribute information of the child 42. The personality of the child 42 may include being sociable, being introverted, being particular about what to eat, being easily bored, and the like. Specifically, in an input screen for the resume information 210, a subjective evaluation of the child 42 seen from the assistance target person 40 may be able to be selectively input.

For example, the matching unit 112 combines the user 20 who has experience rearing a boy with the assistance target person 40 who has a boy. The matching unit 112 combines the user 20 who has a large daily conversation amount with the assistance target person 40 who has a highly sociable child.

Since assistance for the user 20 includes taking care of the child 42 of the assistance target person 40, a possibility that a compatible combination of the child 42 and the user 20 can be made increases by matching an attribute of the child 42 and an attribute of the user 20. The compatible combination allows for continuation of the assistance.

The acquisition unit 102 further acquires environmental information relating to an environment in which a task is performed.

The matching unit 112 generates a combination of the user 20 and the assistance target person 40 by further using the environmental information and an evaluation value of a health risk for the user 20.

The environmental information includes, for example, information indicating load status within an assisting activity area, including a travel section to a home of the assistance target person 40 (e.g., presence or absence of a step, presence or absence of a slope, presence or absence of an elevator and an escalator, and the like). Further, the environmental information includes information indicating barrier-free status of the home of the assistance target person 40 (presence or absence of a step, presence or absence of a stair, presence or absence of a handrail, and the like).

For example, the matching unit 112 may determine a travel section by using an address described in the resume information 210 of the user 20 and an address of the assistance target person 40, and perform matching processing by using a result of determining whether the user 20 is capable of commuting to the home of the assistance target person 40.

For example, when the user 20 has a development risk of osteoporosis (exercise assistance required), the assistance target person 40 with a low load indicated by the environmental information is combined. For example, the matching unit 112 combines the assistance target person 40 whose travel section has no step and no slope but has an elevator and an escalator and whose home is barrier-free, with the user 20 who has a development risk of osteoporosis (exercise assistance required).

In this way, by considering an environment of a workplace, it becomes possible for the user 20 to perform a task safely. It also becomes possible to reduce occurrence of an accident and an injury to the user 20 during a task.

The acquisition unit 102 acquires a remuneration item list 250 in which a remuneration for a task of the user 20 is settled.

The matching unit 112 generates a combination of the user 20 and the assistance target person 40 by further using the remuneration.

A task of the user 20 includes a mutual aid activity. It is assumed that the assistance target person 40 can reduce or offset a remuneration to the user 20 by incorporating a mechanism of providing consideration other than money to the user 20 as a remuneration to the user 20. As described above, since the assistance target person 40 is a single mother or the like having a financial difficulty, it is preferable that a remuneration to the user 20 includes one that does not involve exchange of money.

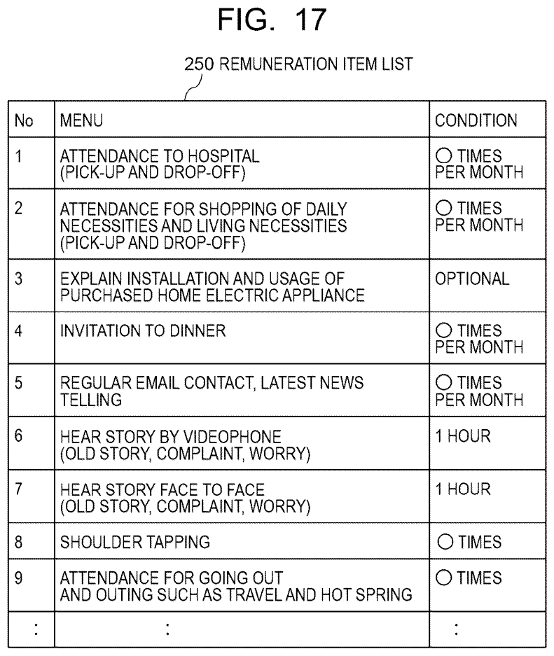

FIG. 17 is a diagram illustrating a data structure example of the remuneration item list 250. As illustrated in the remuneration item list 250, the assistance target person 40 provides the user 20 with a thing that helps the user 20, a thing that pleases the user 20, and a thing that is beneficial to the user 20 as a remuneration.

For example, the remuneration item list 250 may be proposed from the user 20 to the assistance target person 40 and may make the assistance target person 40 to select, may be proposed from the assistance target person 40 to the user 20 and may make the user 20 to select, or may be both. The remuneration item list 250 may be selected from predetermined ones, may be freely proposed by the user 20 or the assistance target person 40, or may be a combination of both.

Moreover, the resume information 210 of the user 20 may include a consideration item such as a medical history and hospital attendance status. Matching may be performed by whether the remuneration item list 250 includes a remuneration being relevant to the consideration item. For example, a remuneration for accompanying in hospital attendance matches the user 20 who needs hospital attendance.

The remuneration item list 250 may be exchanged between the user 20 and the assistance target person 40 when building an agreement on whether to receive assistance. In other words, the user 20 and the assistance target person 40 matched by the matching unit 112 may present the remuneration item list 250 to each other, settle a remuneration, and build an agreement.

In this way, by determining a remuneration as consideration other than money, it becomes possible even for a single mother in financially difficult status to continuously receive assistance. In other words, continuation of utilization by the assistance target person 40 can be encouraged.

Moreover, the matching unit 112 may detect a conversation amount by performing audio processing on a video when the user 20 and the child 42 are together, and determine compatibility. Moreover, the matching unit 112 may perform facial expression analysis and behavior analysis by performing image processing on a video when the user 20 and the child 42 are together, and determine compatibility.

For example, the matching unit 112 may acquire a proportion of an expressionless or silent time, a frequency and a degree of smile, presence or absence and frequency of a gesture such as nodding, a speed of conversation, and a change amount in intonation, and determine compatibility. Alternatively, the matching unit 112 may determine compatibility by determining whether a conversation of the user 20 is easy to understand for the child 42, whether the user 20 is speaking slowly and clearly, and whether the user 20 is using an easy-to-understand word.

The matching unit 112 combines the user 20 and child 42 (assistance target person 40) who have been determined to be compatible. By making a compatible combination, a possibility that continued utilization can be encouraged heightens.

### <Operation Example>

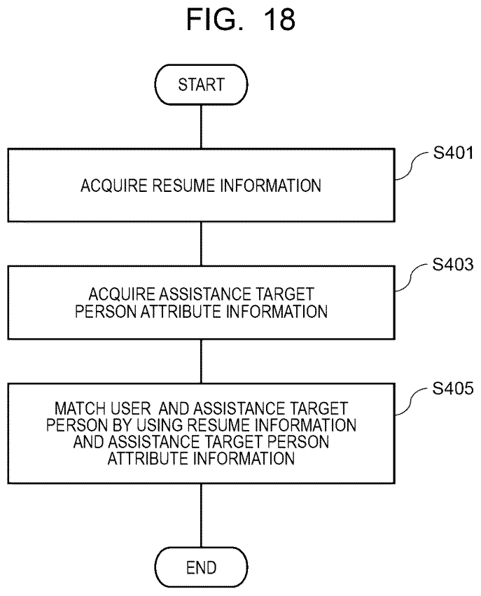

FIG. 18 is a flowchart illustrating an operation example of the work assisting apparatus 100 according to the example embodiment.

First, the acquisition unit 102 acquires the resume information 210 including information relating to an experience of child rearing of the user 20 (step S401). Further, the acquisition unit 102 acquires assistance target person attribute information indicating an attribute of the child 42 (step S403).

The matching unit 112 generates a combination of the user 20, the assistance target person 40, and the child 42 by using the resume information 210 and the assistance target person attribute information (step S405).

For example, the user 20 who has experience rearing a boy is combined with the assistance target person 40 who has a boy.

As described above, according to the present example embodiment, the work assisting apparatus 100 includes the matching unit 112. The acquisition unit 102 acquires assistance target person attribute information indicating an attribute of the child 42. The matching unit 112 generates a combination of the user 20, the assistance target person 40, and the child 42 by using the resume information 210 and the assistance target person attribute information.

In this way, the work assisting apparatus 100 according to the present example embodiment provides an effect similar to the example embodiments described above, a possibility that a compatible combination of the child 42 and the user 20 can be made increases, and it becomes possible to continue assistance for a compatible combination. By promoting continued utilization of assistance, a possibility that work of the assistance target person 40 changes from non-regular work such as a part-timer to full-time work is expanded, and it becomes possible to encourage financial independence of the assistance target person 40.

### Fifth Example Embodiment

The present example embodiment is similar to any of the example embodiments described above, except for having a configuration that regularly monitors a health risk for the user 20, and, when there is a change, reviews assisting work. Since a work assisting apparatus 100 according to the present example embodiment has the same configuration as the work assisting apparatus 100 in FIG. 5, description is given by using FIG. 5. Note that, the configuration according to the present example embodiment may be combined with at least one of configurations according to other example embodiments to an extent that no contradiction occurs.

### <Functional Configuration Example>

An acquisition unit 102 periodically acquires biological information 200 and resume information 210.

When the acquisition unit 102 acquires information, a computation unit 104 computes a health risk for the user 20.

When it is detected that there is a change in health risk, a determination unit 106 updates a candidate for a task that the user 20 is capable of taking charge of.

### <Operation Example>

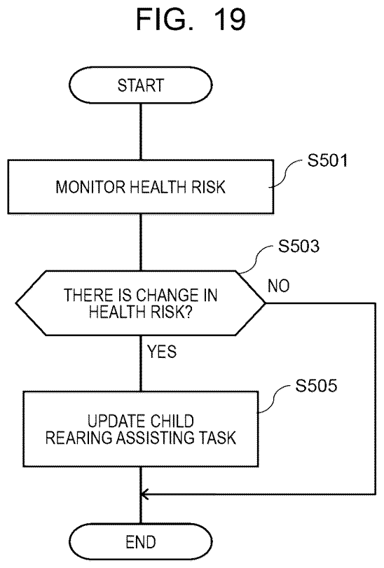

FIG. 19 is a flowchart illustrating an operation example of a work assisting apparatus 100 according to the example embodiment.

First, monitoring of a health risk is regularly performed (step S501). Specifically, the acquisition unit 102 periodically acquires the biological information 200 and the resume information 210 of the user 20, and, when the acquisition unit 102 acquires information, the computation unit 104 computes a health risk for the user 20.

Then, the determination unit 106 determines whether there is a change between the computed health risk and a past health risk for the user 20 (step S503). When there is a change (YES in step S503), the determination unit 106 updates a candidate for a task that the user 20 is capable of taking charge of (step S505). Specifically, processing of at least one of the flowcharts in FIGS. 9, 13, and 15 of the example embodiment described above is executed. When there is no change in the health risk (NO in step S503), step S505 is bypassed and the processing ends.

In the determination processing of a change in the health risk in step S503, when assessing a sudden change, it is determined whether a change amount computed by comparison with only a previous health risk exceeds a certain value. On the other hand, when assessing a slow change, it may be determined whether there is a decreasing trend or an increasing trend in changes in a plurality of amounts of health risks over a certain period of time.

Moreover, since an exercise amount may decrease with age, a learning engine may be generated, and a change in a health risk at an age of the user 20 may be simulated and used for the determination described above. Thereby, there is a possibility that a lead time of searching for an assister replacing the user 20 can be reduced by assessing in advance that a probability that the user 20 becomes unable to perform assistance sufficient for the assistance target person 40 with age. On the other hand, there is a possibility that matching with another of the assistance target persons 40 with a lighter task load (e.g., the number of the children 42 is small, an age is high, a girl, or the like) is performed based on the assessment result in consideration of a health state of the user 20 changing with age, and a task can be assigned.

Moreover, when it is determined in step S503 that there is a change in the health risk (YES in step S503), the determination unit 106 determines, by using a task item list 230 including help information 260, a candidate of a task that the user 20 is capable of taking charge of, and an output processing unit 108 may display the candidate of the task including the assistance information 260 on a display of the user terminal 30 of the user 20.

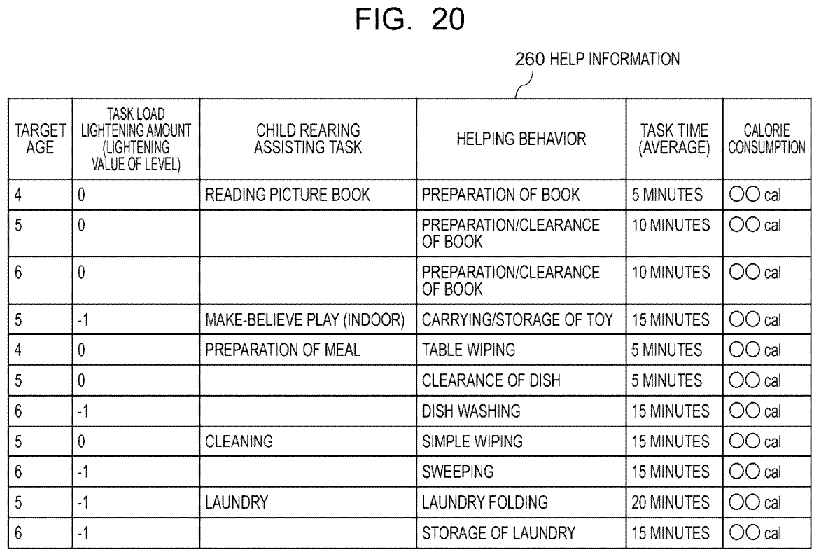

FIG. 20 is a diagram illustrating a data structure example of the help information 260.

In the helping information 260, each task in the task item list 230 in FIG. 8 is further related to a helping behavior of a child and a lightening amount in a task load level due to the helping by the child.

With the help information 260, the assistance target person 40 can previously specify, by using an assistance target person terminal 50, which of the helpings it is possible to provide. Then, the user 20 can select a task after confirming a helping behavior from among candidates of tasks.

As described above, according to the present example embodiment, a configuration is provided in which a health risk for the user 20 is regularly monitored, and, when there is a change, the assisting work is reviewed.

In this way, the work assisting apparatus 100 according to the present example embodiment provides an effect similar to the example embodiments described above, and, further, enables a task with an appropriate load adapted to a change in a health risk for the user 20 due to aging.

Moreover, by performing help of a task by the child 42, a task load on the user 20 can be reduced. Helping serves as a part of discipline for the child 42, and also has the advantage of helping with a decline in physical strength due to aging for the user 20, and a mutual aid activity between an assisting side and an assisted side can be smoothly achieved.

The example embodiments of the present invention have been described above with reference to the drawings, but are exemplifications of the present invention, and various configurations other than those described above can also be adopted.

### <Computation of a Development Risk of Dementia>

For example, in the example embodiment described above, a development risk of dementia is computed based on a deviation between an estimated age of an appearance using a face image of the user 20 and an actual age of the user 20. In another example embodiment, information indicating a frequency of communication in a daily life of the user 20, the number of communication target persons, and a relationship of a communication target person (e.g., whether a person is a relative or another person, and a size of a human network) is acquired, and a development risk of dementia can also be computed by machine learning and deep learning.

### <Facial Expression Analysis and Behavior Analysis>

The biological information 200 includes a face image of user 20.

The acquisition unit 102 acquires a video captured during an online interview with the user 20.

The computation unit 104 performs at least one of facial expression analysis and behavior analysis of the user 20 by using the video, and computes an evaluation value indicating a health risk for the user 20.

For example, richness (such as the number of smiles) of facial expressions of the user 20 of the user 20 may be acquired by facial expression analysis, and an evaluation value of a development risk of dementia of the user 20 may be computed.

According to this configuration, accuracy of an evaluation value of a development risk of dementia of the user 20 can be further improved.

### <Development Risk of Arteriosclerosis>

In the example embodiments described above, a configuration that computes a development risk of dementia from a face image has been described in detail. In another example embodiment, a configuration that computes a development risk of arteriosclerosis from a face image can also be conceived.

For example, the computation unit 104 estimates a blood vessel age by performing image processing on a face image.

Then, the computation unit 104 derives an evaluation value indicating a health risk, based on an actual age acquired from the resume information 210 and an estimated blood vessel age. More specifically, a determined actual age is compared with an estimated age, and, when the actual age exceeds the estimated age, an evaluation value indicating a health risk is determined as a value indicating that there is a development risk of arteriosclerosis. Note that, when the actual age exceeds the estimated age by equal to or more than a certain age, the evaluation value indicating the health risk may be determined as a value indicating that there is a development risk of arteriosclerosis. In this way, the computation unit 104 can estimate, based on the face image and the resume information 210, a health risk such as arteriosclerosis that has a possibility of occurring due to aging of a blood vessel.

Moreover, a task load level set in the task item list 230 may be set based on Metabolic equivalents (METs) indicating exercise intensity. Note that, a plurality of task load levels may be set for one task content. For example, it is such that a task load level set based on calorie consumption and a task load level set based on exercise intensity are managed in association with a specific task.

Then, the determination unit 106 may change a reference task load level to be referred to, according to an evaluation value indicating a health risk, when determining, from among task items in the task item list 230, a candidate of a task that the user 20 is capable of taking charge of. For example, it is such that, when an evaluation value indicating a health risk is a value indicating that there is a development risk of arteriosclerosis, a candidate of a task that the user is capable of taking charge of is determined based on a task load level set based on METs indicating exercise intensity.

Note that, when a task load level to be referred to is changed according to an evaluation value indicating a health risk, types of task load levels to be referred to may be changed to be increase, or may be changed to be decreased. Further, it may be limited in such a way as to refer to only a specific item. In this way, it becomes possible for the determination unit 106 to determine a task item suited to an evaluation value of a health risk for the user 20 as a candidate of a task to present to the user.

Moreover, although a plurality of processes (pieces of processing) are described in order in a plurality of flowcharts used in the above description, an execution order of processes executed in each example embodiment is not limited to the described order. In each example embodiment, an order of illustrated processes can be changed to an extent that causes no problem in terms of content. Moreover, at least one process may be performed by another operating entity, for example, another apparatus or person. Moreover, each of the example embodiments described above can be combined to an extent that content does not contradict.

While the invention of the present application has been described above with reference to the example embodiments, the invention of the present application is not limited to the example embodiments described above. Various changes that can be understood by those skilled in the art can be made to the configuration and details of the invention of the present application within the scope of the present invention.

Note that, when information relating to a user (the user 20, the assistance target person 40, and the child 42) is acquired and utilized in the present invention, this is performed legally.

Some or all of the above-described example embodiments can also be described as, but are not limited to, the following supplementary notes.
1. A work assisting apparatus including:
   an acquisition unit that acquires biological information of a user and resume information of the user;
   a computation unit that computes an evaluation value indicating a health risk for the user, by using the biological information and the resume information that have been acquired; and
   a determination unit that determines, by using the evaluation value indicating the health risk, a candidate of a task to present to the user.
2. The work assisting apparatus according to 1., wherein
   the biological information includes health information indicating a health state of the user.
3. The work assisting apparatus according to 2., wherein
   the health information includes information relating to bone mass.
4. The work assisting apparatus according to 3., wherein
   the evaluation value indicating the health risk includes an evaluation value indicating a development risk of osteoporosis.
5. The work assisting apparatus according to 4., wherein
   the computation unit computes an evaluation value indicating a development risk of osteoporosis by using an age of the user estimated by using the information relating to the bone mass, and an age of the user included in the resume information.
6. The work assisting apparatus according to any one of 1. to 5., wherein
   the biological information includes a face image of the user, and
   the computation unit computes the evaluation value indicating the health risk, based on an age estimated from the face image and the age of the user included in the resume information.
7. The work assisting apparatus according to any one of 1. to 6., wherein
   the determination unit determines an allowable load range of the user, based on the evaluation value indicating the health risk, and determines, based on load information determined for each task, a candidate of the task to present to the user.
8. The work assisting apparatus according to any one of 1. to 7., wherein
   the task includes assisting an assistance target person, and a mutual aid activity.
9. The work assisting apparatus according to 8., wherein
   the acquisition unit acquires target person attribute information indicating an attribute of the assistance target person,
   the work assisting apparatus further including
   a generation unit that generates a task item list, based on the target person attribute information, wherein
   the determination unit determines a candidate of a task to present to the user from among tasks included in the task item list.
10. The work assisting apparatus according to 8, or 9., wherein
   the task includes a task relating to a child of the assistance target person,
   the resume information includes information relating to experience of child rearing of the user, and
   the acquisition unit acquires assistance target person attribute information including an attribute of the child of the assistance target person,
   the work assisting apparatus further including
   a matching unit that generates a combination of the user and the assistance target person by using the resume information and the assistance target person attribute information.
11. The work assisting apparatus according to 10., wherein
   the acquisition unit acquires, from a remuneration item list in which a remuneration for the task of the user is settled, the remuneration selected by the user, and
   the matching unit generates a combination of the user and the assistance target person by further using the selected remuneration.
12. The work assisting apparatus according to 10. or 11., wherein
   the acquisition unit further acquires environmental information relating to an environment in which the task is performed, and
   the matching unit generates a combination of the user and the assistance target person by further using the environmental information and the evaluation value of the health risk of the user.
13. The work assisting apparatus according to any one of 1. to 12., wherein
   the biological information includes a face image of the user,
   the acquisition unit acquires a video captured during an online interview with the user, and
   the computation unit performs at least one of facial expression analysis and behavior analysis of the user by using the video, and computes an evaluation value indicating a health risk for the user.
14. A work assisting method including,
   by one or more computers:
   acquiring biological information of a user and resume information of the user;
   computing an evaluation value indicating a health risk for the user, by using the biological information and the resume information that have been acquired; and determining, by using the evaluation value indicating the health risk, a candidate of a
   task to present to the user.
15. The work assisting method according to 14., wherein
   the biological information includes health information indicating a health state of the user.
16. The work assisting method according to 15., wherein
   the health information includes information relating to bone mass.
17. The work assisting method according to 16., wherein
   the evaluation value indicating the health risk includes an evaluation value indicating a development risk of osteoporosis.
18. The work assisting method according to 17., further including,
   by one or more computers,
   computing an evaluation value indicating a development risk of osteoporosis by using an age of the user estimated by using the information relating to the bone mass, and an age of the user included in the resume information.
19. The work assisting method according to any one of 14. to 18., wherein
   the biological information includes a face image of the user,
   the work assisting method further including,
   by one or more computers,
   computing the evaluation value indicating the health risk, based on an age estimated from the face image and the age of the user included in the resume information.
20. The work assisting method according to any one of 14. to 19., further including,
   by one or more computers,
   determining an allowable load range of the user, based on the evaluation value indicating the health risk, and determining, based on load information determined for each task, a candidate of the task to present to the user.
21 The work assisting method according to any one of 14. to 20., wherein
   the task includes assisting an assistance target person, and a mutual aid activity.
22. The work assisting method according to 21., further including, by one or more computers:
   acquiring target person attribute information indicating an attribute of the assistance target person;
   generating a task item list, based on the target person attribute information; and
   determining a candidate of a task to present to the user from among tasks included in the task item list.
23 The work assisting method according to 21. or 22., wherein
   the task includes a task relating to a child of the assistance target person, and
   the resume information includes information relating to experience of child rearing of the user,
   the work assisting method further including,
   by one or more computers:
   acquiring assistance target person attribute information including an attribute of the child of the assistance target person; and
   generating a combination of the user and the assistance target person by using the resume information and the assistance target person attribute information.
24. The work assisting method according to 23., further including,
   by one or more computers:
   acquiring, from a remuneration item list in which a remuneration for the task of the user is settled, the remuneration selected by the user; and
   generating a combination of the user and the assistance target person by further using the selected remuneration.
25 The work assisting method according to 23. or 24., further including,
   by one or more computers:
   further acquiring environmental information relating to an environment in which the task is performed; and
   generating a combination of the user and the assistance target person by further using the environmental information and the evaluation value of the health risk of the user.
26. The work assisting method according to any one of 14. to 25., wherein
   the biological information includes a face image of the user,
   the work assisting method further including,
   by one or more computers:
   acquiring a video captured during an online interview with the user; and
performing at least one of facial expression analysis and behavior analysis of the user by using the video, and computing an evaluation value indicating a health risk for the user.
27. A program for causing a computer to execute:
   a procedure of acquiring biological information of a user and resume information of the user;
   a procedure of computing an evaluation value indicating a health risk for the user, by using the biological information and the resume information that have been acquired; and
   a procedure of determining, by using the evaluation value indicating the health risk, a candidate of a task to present to the user.
28. The program according to 27., wherein
   the biological information includes health information indicating a health state of the user.
29. The program according to 28., wherein
   the health information includes information relating to bone mass.
30. The program according to 29., wherein
   the evaluation value indicating the health risk includes an evaluation value indicating a development risk of osteoporosis.
31. The program according to 30., wherein
   in the procedure of computing, an evaluation value indicating a development risk of osteoporosis is computed by using an age of the user estimated by using the information relating to the bone mass, and an age of the user included in the resume information.
32. The program according to any one of 27. to 31., wherein
   the biological information includes a face image of the user, and
   in the procedure of computing, the evaluation value indicating the health risk is computed, based on an age estimated from the face image and the age of the user included in the resume information.
33. The program according to any one of 27. to 32., wherein
   in the procedure of determining, an allowable load range of the user is determined based on the evaluation value indicating the health risk, and a candidate of the task to present to the user is determined, based on load information determined for each task.
34. The program according to any one of 27. to 33., wherein
   the task includes assisting an assistance target person, and a mutual aid activity.
35. The program according to 34., wherein
   in the procedure of acquiring, target person attribute information indicating an attribute of the assistance target person is acquired,
   the program causes a computer to execute a procedure of generating a task item list, based on the target person attribute information, and
   in the procedure of determining, a candidate of a task to present to the user is determined from among tasks included in the task item list.
36. The program according to 34. or 35., wherein
   the task includes a task relating to a child of the assistance target person,
   the resume information includes information relating to experience of child rearing of the user,
   in the procedure of acquiring, assistance target person attribute information including an attribute of the child of the assistance target person is acquired, and
   the program causes a computer to execute a procedure of generating a combination of the user and the assistance target person by using the resume information and the assistance target person attribute information.
37. The program according to 36., wherein
   in the procedure of acquiring, from a remuneration item list in which a remuneration for the task of the user is settled, the remuneration selected by the user is acquired, and
   in the procedure of generating a combination, a combination of the user and the assistance target person is generated by further using the selected remuneration.
38. The program according to 36. or 37., wherein
   in the procedure of acquiring, environmental information relating to an environment in which the task is performed is further acquired, and
   in the procedure of generating a combination, a combination of the user and the assistance target person is generated by further using the environmental information and the evaluation value of the health risk of the user.
39. The program according to any one of 27. to 38., wherein
   the biological information includes a face image of the user,
   in the procedure of acquiring, a video captured during an online interview with the user is acquired, and
   in the procedure of computing, at least one of facial expression analysis and behavior analysis of the user is performed by using the video, and an evaluation value indicating a health risk for the user is computed.
40. A computer-readable storage medium storing a program that causes a computer to execute:
   a procedure of acquiring biological information of a user and resume information of the user;
   a procedure of computing an evaluation value indicating a health risk for the user, by using the biological information and the resume information that have been acquired; and
   a procedure of determining, by using the evaluation value indicating the health risk, a candidate of a task to present to the user.
41. The computer-readable storage medium storing a program according to 40., wherein
   the biological information includes health information indicating a health state of the user.
42. The computer-readable storage medium storing a program according to 41., wherein
   the health information includes information relating to bone mass.
43. The computer-readable storage medium storing a program according to 42., wherein
   the evaluation value indicating the health risk includes an evaluation value indicating a development risk of osteoporosis.
44. The computer-readable storage medium storing a program according to 43., wherein
   in the procedure of computing, an evaluation value indicating a development risk of osteoporosis is computed by using an age of the user estimated by using the information relating to the bone mass, and an age of the user included in the resume information.
45. The computer-readable storage medium storing a program according to any one of 40. to 44., wherein
   the biological information includes a face image of the user, and
   in the procedure of computing, the evaluation value indicating the health risk is computed, based on an age estimated from the face image and the age of the user included in the resume information.
46. The computer-readable storage medium storing a program according to any one of 40. to 45., wherein
   in the procedure of determining, an allowable load range of the user is determined based on the evaluation value indicating the health risk, and a candidate of the task to present to the user is determined, based on load information determined for each task.
47. The computer-readable storage medium storing a program according to any one of 40. to 4646., wherein
   the task includes assisting an assistance target person, and a mutual aid activity.
48. The computer-readable storage medium storing a program according to 47., wherein
   in the procedure of acquiring, target person attribute information indicating an attribute of the assistance target person is acquired,
   the program causes a computer to execute a procedure of generating a task item list, based on the target person attribute information, and
   in the procedure of determining, a candidate of a task to present to the user is determined from among tasks included in the task item list.
49. The computer-readable storage medium storing a program according to 47. or 48., wherein
   the task includes a task relating to a child of the assistance target person,
   the resume information includes information relating to experience of child rearing of the user,
   in the procedure of acquiring, assistance target person attribute information including an attribute of the child of the assistance target person is acquired, and
   the program causes a computer to execute a procedure of generating a combination of the user and the assistance target person by using the resume information and the assistance target person attribute information.
50. The computer-readable storage medium storing a program according to 49., wherein
   in the procedure of acquiring, from a remuneration item list in which a remuneration for the task of the user is settled, the remuneration selected by the user is acquired, and
   in the procedure of generating a combination, a combination of the user and the assistance target person is generated by further using the selected remuneration.
51. The computer-readable storage medium storing a program according to 49. or 50., wherein
   in the procedure of acquiring, environmental information relating to an environment in which the task is performed is further acquired, and
   in the procedure of generating a combination, a combination of the user and the assistance target person is generated by further using the environmental information and the evaluation value of the health risk of the user.
52. The computer-readable storage medium storing a program according to any one of 40. to 51., wherein
   the biological information includes a face image of the user,
   in the procedure of acquiring, a video captured during an online interview with the user is acquired, and
   in the procedure of computing, at least one of facial expression analysis and behavior analysis of the user is performed by using the video, and an evaluation value indicating a health risk for the user is computed.

### REFERENCE SIGNS LIST

1 Work assisting system
3 Communication network
20 User
30 User terminal
32 Wearable terminal
40 Assistance target person
50 Assistance target person terminal
100 Work assisting apparatus
102 Acquisition unit
104 Computation unit
106 Determination unit
108 Output processing unit
112 Matching unit
120 Storage apparatus
200 Biological information
210 Resume information
220) Health risk information
230) Task item list
232 Task item list
250 Remuneration item list
260 Help information
300 Task candidate screen
302 Check box
1000 Computer
1010 Bus
1020 Processor
1030 Memory
1040 Storage device
1050 Input/output interface
1060 Network interface

What is claimed is:

1. A work assisting apparatus comprising:
at least one memory store instructions; and
at least one processor configured to execute the instructions to:
acquire biological information of a user and resume information of the user;
compute an evaluation value indicating a health risk for the user, by using the biological information and the resume information that have been acquired; and
determine, by using the evaluation value indicating the health risk, a candidate of a task to present to the user.

2. The work assisting apparatus according to claim 1, wherein
the biological information includes health information indicating a health state of the user.

3. The work assisting apparatus according to claim 2, wherein
the health information includes information relating to bone mass.

4. The work assisting apparatus according to claim 3, wherein
the evaluation value indicating the health risk includes an evaluation value indicating a development risk of osteoporosis.

5. The work assisting apparatus according to claim 4, wherein
the at least one processor is further configured to execute the instructions to
compute an evaluation value indicating a development risk of osteoporosis by using an age of the user estimated by using the information relating to the bone mass, and an age of the user included in the resume information.

6. The work assisting apparatus according to claim 1, wherein
the biological information includes a face image of the user, and
the at least one processor is further configured to execute the instructions to
compute the evaluation value indicating the health risk, based on an age estimated from the face image and the age of the user included in the resume information.

7. The work assisting apparatus according to claim 1, wherein
the at least one processor is further configured to execute the instructions to
determine an allowable load range of the user, based on the evaluation value indicating the health risk, and determine, based on load information determined for each task, a candidate of the task to present to the user.

8. The work assisting apparatus according to claim 1, wherein
the task includes assisting an assistance target person, and a mutual aid activity.

9. The work assisting apparatus according to claim 8, wherein
the at least one processor is further configured to execute the instructions to:
acquire target person attribute information indicating an attribute of the assistance target person;
generate a task item list, based on the target person attribute information; and information, wherein
determine a candidate of a task to present to the user from among tasks included in the task item list.

10. The work assisting apparatus according to claim 8, wherein
the task includes a task relating to a child of the assistance target person,
the resume information includes information relating to experience of child rearing of the user, and
the at least one processor is further configured to execute the instructions to:
acquire assistance target person attribute information including an attribute of the child of the assistance target person; and
generate a combination of the user and the assistance target person by using the resume information and the assistance target person attribute information.

11. The work assisting apparatus according to claim 10, wherein
the at least one processor is further configured to execute the instructions to:
acquire, from a remuneration item list in which a remuneration for the task of the user is settled, the remuneration selected by the user; and
generate a combination of the user and the assistance target person by further using the selected remuneration.

12. The work assisting apparatus according to claim 10, wherein
the at least one processor is further configured to execute the instructions to:
further acquire environmental information relating to an environment in which the task is performed; and
generate a combination of the user and the assistance target person by further using the environmental information and the evaluation value of the health risk of the user.

13. The work assisting apparatus according to claim 1, wherein
the biological information includes a face image of the user,
the at least one processor is further configured to execute the instructions to:
acquire a video captured during an online interview with the user; and
perform at least one of facial expression analysis and behavior analysis of the user by processing the video to estimate facial expression and behavior of the user, and compute an evaluation value indicating a health risk for the user.
*Based on the descriptions in paragraphs. 0168, 0169, 0187, and 0188 in the WO specifications.

14. A work assisting method comprising,
by one or more computers:
acquiring biological information of a user and resume information of the user;
computing an evaluation value indicating a health risk for the user, by using the biological information and the resume information that have been acquired; and
determining, by using the evaluation value indicating the health risk, a candidate of a task to present to the user.

15. A non-transitory computer-readable storage medium storing a program for causing a computer to execute:
a procedure of acquiring biological information of a user and resume information of the user;
a procedure of computing an evaluation value indicating a health risk for the user, by using the biological information and the resume information that have been acquired; and
a procedure of determining, by using the evaluation value indicating the health risk, a candidate of a task to present to the user.

16. The work assisting apparatus according to claim 1, wherein
the evaluation value is computed using machine learning, and
the determined candidate of the task is used to have the user make decision concerning the work.
